(19) United States
(12) Patent Application Publication
(10) Pub. No.: US 2026/0114994 A1
(43) Pub. Date: Apr. 30, 2026

(54) PROSTHETIC VALVE LEAFLET MATERIAL, VALVE LEAFLET, VALVE AND PREPARATION METHOD

(71) Applicant: VENUS MEDTECH (HANGZHOU) INC., Hangzhou (CN)

(72) Inventors: Lichao Wang, Hangzhou (CN); Xing Wei, Hangzhou (CN); Dajun Kuang, Hangzhou (CN); Manman Zhao, Hangzhou (CN); Lingfeng Yang, Hangzhou (CN)

(21) Appl. No.: 19/433,012

(22) Filed: Dec. 25, 2025

Related U.S. Application Data
(63) Continuation-in-part of application No. PCT/CN2023/112864, filed on Aug. 14, 2023.

(30) Foreign Application Priority Data
Jun. 27, 2023 (CN) 202310770514.4

(51) Int. Cl. A61F 2/24

(52) U.S. Cl. CPC A61F 2/2418; A61F 2/2415; A61F 2230/0069

(57) ABSTRACT

The present disclosure discloses a prosthetic valve leaflet material, a valve leaflet, a valve, and a preparation method thereof. The prosthetic valve leaflet material is made of polyurethane and includes an inner layer and surface layers located on opposite sides of the inner layer in the thickness direction, wherein the elastic modulus of the surface-layer polyurethane is greater than that of the inner-layer polyurethane. The valve leaflet material simultaneously takes into account fluid dynamics, biostability, biocompatibility, creep resistance, and fatigue resistance, achieving the performance required of the heart valves.

### CROSS-REFERENCE TO RELATED APPLICATIONS

The present disclosure is a Continuation-In-Part Application of PCT Application No. PCT/CN2023/112864, filed on Aug. 14, 2023, which claims priority to Chinese Patent Application No. 202310770514.4, filed on Jun. 27, 2023, the entire contents of which are hereby incorporated by reference.

### TECHNICAL FIELD

The present disclosure relates to the technical field of medical materials, and in particular to a prosthetic valve leaflet material, a valve leaflet, a valve and a preparation method thereof.

### BACKGROUND

Polyurethane materials have been widely studied for use as prosthetic valve leaflet materials, yet they still present several challenges in this application. Polyurethane molecular chains consist of hard and soft segments. In valve applications, the common failure resulting from performance degradation mainly include oxidative degradation of the soft segments, hydrolytic degradation, fatigue (including tearing caused by bending and damage caused by stress concentration from surface defects), and creep elongation.

In terms of the molecular structure of polyurethane materials, a relatively high hard segment content (or a high urea linkage content) can effectively increase the interaction forces between molecular chains, resulting in a denser material structure and improved mechanical properties. Meanwhile, as the hard segment content is relatively high in proportion and the soft segment content is relatively low, the risks of oxidation and hydrolysis caused by the soft segment's own structure are also reduced. This leads to significant improvements in the material's biostability, fatigue resistance, creep resistance, and biocompatibility. However, when the hard segment content is excessively high, the material's elastic modulus (also known as Young's modulus) also increases, making it difficult to meet the hydrodynamic performance requirements for valve leaflet movement when used as a valve material.

### Technical Problems

To summarize, polyurethane, when used as the valve leaflet material, finds it difficult to simultaneously balance hydrodynamic performance, biostability, biocompatibility, creep resistance, and fatigue resistance to meet all the performance requirements for heart valves. Therefore, no commercial polyurethane material has yet been successfully applied to heart valves.

### SUMMARY

The present disclosure provides a synthetic valve leaflet material that simultaneously balances fluid mechanics, biostability, biocompatibility, creep resistance and fatigue resistance, thereby achieving the performance required for heart valves.

A prosthetic valve leaflet material is made of polyurethane and includes an inner layer and surface layers located on two opposite sides of the inner layer in the thickness direction, wherein the elastic modulus of the surface-layer polyurethane is greater than the elastic modulus of the inner-layer polyurethane.

In the following, several alternatives are provided, but merely as further additions or preferences, instead of as further limitations to the above-mentioned technical solution. Without technical or logical contradiction, the alternatives can be combined with the above-mentioned technical solution, individually or in combination.

Optionally, the elastic modulus of the surface-layer polyurethane is 10-60 MPa greater than that of the inner-layer polyurethane. Preferably, the difference is 10-30 MPa.

Optionally, the elastic modulus of the surface-layer polyurethane is 30-100 MPa, preferably 40-60 MPa.

Optionally, the elastic modulus of the inner-layer polyurethane is 5-30 MPa, preferably 20-30 MPa.

Optionally, the oxygen permeability of the surface-layer polyurethane is ≤9*10⁹ (cm²/s*cm*Hg).

Optionally, the molecular chain structures of the surface-layer polyurethane and the inner-layer polyurethane shall satisfy at least one of the following conditions.

a. The hard segment content of the inner-layer polyurethane is lower than that of the surface-layer polyurethane.

b. The inner-layer polyurethane is prepared using an alcohol-based chain extender, and the surface-layer polyurethane is prepared using an amine-containing chain extender.

c. The surface-layer polyurethane has a higher crosslink density compared to the inner-layer polyurethane.

Optionally, the molecular chain structure of the surface-layer polyurethane includes hard segments and soft segments, wherein the hard segment includes first chain units formed from isocyanate and second chain units formed from a chain extender.

The isocyanate is at least one selected from the following: toluene diisocyanate (TDI), hexamethylene diisocyanate (HDI), diphenylmethane diisocyanate (MDI), naphthalene diisocyanate (NDI), p-phenylene diisocyanate (PPDI), isophorone diisocyanate (IPDI), xylylene diisocyanate (XDI), and triphenylmethane triisocyanate.

The chain extender is at least one selected from the following: ethylene glycol, propylene glycol, butanediol, pentanediol, hexanediol, octanediol, ethylenediamine, propylenediamine, butanediamine, pentanediamine, hexanediamine, octanediamine, and 1,3-bis(4-hydroxybutyl)-1,1,3,3-tetramethyldisiloxane (BHTD).

The soft segment raw material is at least one selected from the following: Dutral T5651, Dutral T5652, polyhexamethylene oxide (PHMO), polytetramethylene glycol (PTMO), adipic acid polyester diol, succinic acid polyester diol and polydimethylsiloxane diol.

Optionally, in the molecular chain structure of the surface-layer polyurethane, the isocyanate may be one of the following: diphenylmethane diisocyanate, toluene diisocyanate, and hexamethylene diisocyanate.

Optionally, in the molecular chain structure of the surface-layer polyurethane, the isocyanate is diphenylmethane diisocyanate.

Optionally, in the molecular chain structure of the surface-layer polyurethane, the chain extender is at least one of ethylenediamine and 1,3-bis(4-hydroxybutyl)-1, 1,3,3-tetramethyldisiloxane.

Optionally, in the molecular chain structure of the surface-layer polyurethane, the chain extender includes ethylenediamine and 1,3-bis(4-hydroxybutyl)-1,1,3,3-tetramethyldisiloxane.

Optionally, in the molecular chain structure of the surface-layer polyurethane, the chain extender includes ethylenediamine and 1,3-bis(4-hydroxybutyl)-1,1,3,3-tetramethyldisiloxane, with a molar ratio of ethylenediamine to 1,3-bis(4-hydroxybutyl)-1,1,3,3-tetramethyldisiloxane 1:0.9 to 1:1.1.

Optionally, in the molecular chain structure of the surface-layer polyurethane, the soft segment raw material is at least one of polydimethylsiloxane and polyhexamethylene oxide.

Optionally, in the molecular chain structure of the surface-layer polyurethane, the soft segment raw material includes polydimethylsiloxane and polyhexamethylene oxide.

Optionally, in the molecular chain structure of the surface-layer polyurethane, the soft segment raw material includes polydimethylsiloxane and polyhexamethylene oxide, and the mass ratio of polydimethylsiloxane to polyhexamethylene oxide is 3-6:1.

Optionally, the molecular chain structure of the inner-layer polyurethane includes hard segments and soft segments, wherein the hard segment includes first chain units formed from isocyanate and second chain units formed from a chain extender.

The isocyanate is at least one selected from the following: toluene diisocyanate (TDI), hexamethylene diisocyanate (HDI), diphenylmethane diisocyanate (MDI), naphthalene diisocyanate (NDI), p-phenylene diisocyanate (PPDI), isophorone diisocyanate (IPDI), xylylene diisocyanate (XDI), and triphenylmethane triisocyanate.

The chain extender is at least one of the following: ethylene glycol, propylene glycol, butanediol, pentanediol, hexanediol, octanediol, ethylenediamine, propylenediamine, butanediamine, pentanediamine, hexanediamine, octanediamine, and 1,3-bis(4-hydroxybutyl)-1,1,3,3-tetramethyldisiloxane (BHTD).

The soft segment raw material is at least one selected from the following: Dutral T5651, Dutral T5652, polyhexamethylene oxide (PHMO), polytetramethylene glycol (PTMO), adipic acid polyester diol, succinic acid polyester diol and polydimethylsiloxane diol.

Optionally, in the molecular chain structure of the inner-layer polyurethane, the isocyanate is one selected from diphenylmethane diisocyanate, toluene diisocyanate, and hexamethylene diisocyanate.

Optionally, in the molecular chain structure of the inner-layer polyurethane, the isocyanate is diphenylmethane diisocyanate.

Optionally, in the molecular chain structure of the inner-layer polyurethane, the chain extender is at least one selected from ethylenediamine and 1,3-bis(4-hydroxybutyl)-1,1,3,3-tetramethyldisiloxane.

Optionally, in the molecular chain structure of the inner-layer polyurethane, the chain extender includes ethylenediamine and 1,3-bis(4-hydroxybutyl)-1,1,3,3-tetramethyldisiloxane.

Optionally, in the molecular chain structure of the inner-layer polyurethane, the chain extender includes ethylenediamine and 1,3-bis(4-hydroxybutyl)-1,1,3,3-tetramethyldisiloxane, with the molar ratio of ethylenediamine to 1,3-bis(4-hydroxybutyl)-1,1,3,3-tetramethyldisiloxane 1:0.9 to 1:1.1.

Optionally, in the molecular chain structure of the inner-layer polyurethane, the soft segment raw material is at least one of polydimethylsiloxane and polyhexamethylene oxide.

Optionally, in the molecular chain structure of the inner-layer polyurethane, the soft segment raw material includes polydimethylsiloxane and polyhexamethylene oxide.

Optionally, in the molecular chain structure of the inner-layer polyurethane, the soft segment raw material includes polydimethylsiloxane and polyhexamethylene oxide, and the mass ratio of polydimethylsiloxane to polyhexamethylene oxide is 3:1 to 6:1.

Optionally, the hard segment content of the surface-layer polyurethane and the inner-layer polyurethane is 30%-60%.

Optionally, the hard segment content of the surface-layer polyurethane and the inner-layer polyurethane is 35%-60%.

Optionally, the isocyanate index of both the surface-layer polyurethane and the inner-layer polyurethane is 1.0-1.1.

Optionally, the isocyanate index of both the surface-layer polyurethane and the inner-layer polyurethane is 1.0-1.05.

Optionally, the isocyanate index of both the surface-layer polyurethane and the inner-layer polyurethane is 1.0-1.02.

Optionally, the thickness of the prosthetic valve leaflet material is 75-210 μm.

Optionally, the thickness of the prosthetic valve leaflet material is 80-210 μm, preferably 130-200 μm.

Optionally, the thickness of the inner-layer polyurethane is 70-170 μm, and the thickness of the surface-layer polyurethane is 5-40 μm.

The design of the surface-layer polyurethane material provides biostability and biocompatibility for the valve leaflet material. Meanwhile, the strong interaction forces between the internal soft and hard segments restrict the movement of the soft segment, reduce its water and oxygen transmission capacity, protect the inner-layer polyurethane, and enhance the overall biostability of the valve leaflet. The inner-layer polyurethane material is designed to achieve the hydrodynamic performance of the valve leaflet.

Since the inner-layer polyurethane accounts for a higher proportion in the thickness direction, it directly determines the mechanical properties of the valve leaflet.

Optionally, the thickness of the inner-layer polyurethane is 70-170 μm, and the thickness of the surface-layer polyurethane is 10-40 μm.

Optionally, the thickness of the inner-layer polyurethane is 70-150 μm, and the thickness of the surface-layer polyurethane is 5-30 μm.

Optionally, the inner layer includes at least one first sub-layer along the thickness direction, wherein each first sub-layer is made of the same type of polyurethane, or the elastic modulus of each first sub-layer gradually increases by a predetermined interval from the inside to the outside in the thickness direction.

Modulation of elastic modulus may be achieved through varying hard segments, different chain extenders, or a combination of both. During valve opening and closing, differences in elastic modulus across the thickness induce shear forces between layers, affecting interlayer bonding strength. These shear forces correlate directly with elastic modulus variations. The gradual increase in elastic modulus disperses such shear forces, further improving the valve leaflet's durability.

Optionally, the elastic modulus of each first sub-layer increases gradually at an interval of 5-10 MPa.

Optionally, the inner layer includes 1 to 6 first sub-layers, with each first sub-layer divided into at least two groups. First sub-layers within the same group share identical elastic modulus, and from the inside to the outside in the thickness direction, the elastic modulus of first sub-layers within each group gradually increases by a predetermined interval.

Optionally, the surface layers include at least one second sub-layer along the thickness direction, wherein each second sub-layer is made of the same type of polyurethane, or the elastic modulus of each second sub-layer gradually increases by a predetermined interval from the inside to the outside in the thickness direction.

Modulation of elastic modulus may be achieved through varying hard segments, different chain extenders, or a combination of both. For the outermost layer of the surface, elastic modulus can be enhanced through surface modification or surface crosslinking. During valve opening and closing, differences in elastic modulus across the thickness induce shear forces between layers, affecting interlayer bonding strength. These shear forces correlate directly with elastic modulus variations. The gradual increase in elastic modulus disperses such shear forces, further improving valve leaflet's durability.

Optionally, the elastic modulus of each second sub-layer gradually increases at an interval of 5-10 MPa.

Optionally, the surface layer includes 2 to 4 second sub-layers, each of which is divided into at least two groups. Within the same group, the elastic modulus of each secondary sub-layer is identical, and the elastic modulus of each group of the second sub-layers gradually increases from the inside to the outside in the thickness direction at intervals of a predetermined value.

Optionally, the hard segment content of the inner-layer polyurethane is lower than the hard segment content of the surface-layer polyurethane.

Optionally, the hard segment content of the surface-layer polyurethane is 45%-60%, and the hard segment content of the inner-layer polyurethane is 30%-45%.

Optionally, the hard segments in the molecular chain structures of the surface-layer polyurethane and the inner-layer polyurethane are modified in one of the following ways:
A. introducing fluorine-containing side chains; and
B. introducing a chain extender containing POSS (Polyhedral Oligomeric Silsesquioxane) groups.

Optionally, the introduction of fluorine-containing side chains is achieved by:
adding a fluorine-containing diisocyanate with a mass fraction of 3% to 10% to the synthetic raw material of the polyurethane.

Optionally, the introduction of POSS groups is achieved by:
adding a POSS-containing diol chain extender with a mass fraction of 1% to 3% to the synthetic raw materials of the polyurethane.

Optionally, the inner-layer polyurethane is prepared by an alcohol-based chain extender, and the surface-layer polyurethane is prepared by an amine-containing chain extender.

Optionally, the alcohol-based chain extender is at least one of the following: ethylene glycol, propylene glycol, butanediol, pentanediol, hexanediol, octanediol, and 1,3-bis(4-hydroxybutyl)-1,1,3,3-tetramethyldisiloxane (BHTD).

Optionally, the amine-based chain extender is at least one of the following: ethylenediamine, propylenediamine, butylenediamine, pentamethylenediamine, hexamethylenediamine, and octanediamine.

Optionally, the alcohol-based chain extender is butanediol.

Optionally, the amine-based chain extender is ethylenediamine.

Optionally, the alcohol-based chain extender is butanediol, and the amine-based chain extender is ethylenediamine.

Optionally, the surface-layer polyurethane undergoes crosslinking modification.

Optionally, the crosslinking modification of the surface-layer polyurethane is carried out by radiation crosslinking or chemical crosslinking.

The present disclosure further provides a valve leaflet made of the prosthetic valve leaflet material.

The present disclosure further provides a valve, including:
a stent, with a blood flow channel defined therein; and
one or more valve leaflets made of the prosthetic valve leaflet material.

The present disclosure further provides a method for preparing the prosthetic valve leaflet material, including:
Step 1: immersing a mold in a first surface-layer polyurethane solution, maintaining it for a predetermined time before removal, and drying it at 30-80° C. under an inert gas atmosphere to form a film.

The above Step 1 is repeated 1 to 9 times to form a first surface layer of the prosthetic valve leaflet material.

Step 2: immersing the mold in an inner-layer polyurethane solution, maintaining it for a predetermined time before removal, and drying it at 30-80° C. under an inert gas atmosphere to form a film.

The above Step 2 is repeated 1 to 9 times to form an inner layer of the prosthetic valve leaflet material.

Step 3: fitting a stent onto the mold, immersing the assembly in a second surface-layer polyurethane solution, maintaining it for a predetermined time before removal, and drying it at 30-80° C. under an inert gas atmosphere to form a film.

The above Step 3 is repeated 1 to 9 times to form a second surface layer of the prosthetic valve leaflet material.

Step 4: performing demolding by immersing the mold in water to obtain the prosthetic valve leaflet material.

Several optional methods are further provided below, but they are not intended to be additional limitations on the above-mentioned overall solution. They are merely further supplements or optimizations. Under the premise that there are no technical or logical contradictions, each optional method can be combined separately for the above-mentioned overall solution, or multiple optional methods can be combined.

Optionally, the hard segment content of the surface-layer polyurethane is 45-60%, and the hard segment content of the inner-layer polyurethane is 30-45%.

Optionally, the inner-layer polyurethane is prepared using an alcohol-based chain extender, and the surface-layer polyurethane is prepared using an amine-containing chain extender.

Optionally, the alcohol-based chain extender is at least one of ethylene glycol, propylene glycol, butanediol, pentanediol, hexanediol, octanediol, and 1,3-bis(4-hydroxybutyl)-1,1,3,3-tetramethyldisiloxane (BHTD).

Optionally, the amine-based chain extender is at least one of the following: ethylenediamine, propylenediamine, butylenediamine, pentamethylenediamine, hexamethylenediamine, and octanediamine.

Optionally, the concentration of the polyurethane solution in the first surface layer is 10-30% (w/v), and the concentration of the polyurethane solution in the second surface layer is 5-15% (w/v).

Optionally, the concentration of the inner-layer polyurethane solution is 10-30% (w/v).

Optionally, the solvents of the first surface-layer polyurethane solution, the second surface-layer polyurethane solution, and the inner-layer polyurethane solution are each selected from one of the following: N,N-dimethylacetamide (DMAc), dimethylformamide (DMF), dimethyl sulfoxide (DMSO), toluene, and tetrahydrofuran (THF).

Optionally, in Step 1, the drying time is 1 to 6 h.

Optionally, in Step 2, the drying time is 1 to 6 h.

Optionally, in Step 3, the drying time is 1 to 12 h.

Optionally, in Step 1, the predetermined maintaining time in the first surface-layer polyurethane solution is 5 to 10 s.

Optionally, in Step 2, the predetermined maintaining time in the inner-layer polyurethane solution is 5 to 10 s.

Optionally, in Step 3, the predetermined maintaining time in the second surface-layer polyurethane solution is 5 to 10 s.

The present disclosure further provides a method for preparing a prosthetic valve leaflet material, including:
Step 1, preparing a film with a thickness of 80 to 210 μm from polyurethane having an elastic modulus of 5-30 MPa; and
Step 2: applying an isocyanate solution containing a catalyst onto the surface of the film, and reacting at 50-90° C. for 3-5 h to obtain the prosthetic valve leaflet material.

In the following, several alternatives are provided, but merely as further additions or preferences, instead of as further limitations to the above-mentioned technical solution. Without technical or logical contradiction, the alternatives can be combined with the above-mentioned technical solution, individually or in combination.

Optionally, the molecular chain structure of the polyurethane includes hard and soft segments, wherein the hard segments include first chain units formed from isocyanate and second chain units formed from a chain extender.

The isocyanate is at least one of the following: toluene diisocyanate (TDI), hexamethylene diisocyanate (HDI), diphenylmethane diisocyanate (MDI), naphthalene diisocyanate (NDI), p-phenylene diisocyanate (PPDI), isophorone diisocyanate (IPDI), xylylene diisocyanate (XDI), and triphenylmethane triisocyanate.

The chain extender is at least one of the following: ethylene glycol, propylene glycol, butanediol, pentanediol, hexanediol, octanediol, ethylenediamine, propylenediamine, butanediamine, pentanediamine, hexanediamine, octanediamine, and 1,3-bis(4-hydroxybutyl)-1,1,3,3-tetramethyldisiloxane (BHTD).

The soft segment raw material is at least one of the following: Dutral T5651, Dutral T5652, polyhexamethylene oxide (PHMO), polytetramethylene glycol (PTMO), adipic acid polyester diol, succinic acid polyester diol, and polydimethylsiloxane diol.

Optionally, in the molecular chain structure of the polyurethane, the isocyanate is one of diphenylmethane diisocyanate, toluene diisocyanate, and hexamethylene diisocyanate.

Optionally, in the molecular chain structure of the polyurethane, the isocyanate is diphenylmethane diisocyanate.

Optionally, in the molecular chain structure of the polyurethane, the chain extender is at least one of ethylenediamine and 1,3-bis(4-hydroxybutyl)-1,1,3,3-tetramethyldisiloxane.

Optionally, in the molecular chain structure of the polyurethane, the chain extender includes ethylenediamine and 1,3-bis(4-hydroxybutyl)-1,1,3,3-tetramethyldisiloxane.

Optionally, in the molecular chain structure of the polyurethane, the chain extender includes ethylenediamine and 1,3-bis(4-hydroxybutyl)-1,1,3,3-tetramethyldisiloxane, and the molar ratio of ethylenediamine to 1,3-bis(4-hydroxybutyl)-1,1,3,3-tetramethyldisiloxane is 1:0.9 to 1:1.1.

Optionally, in the molecular chain structure of the polyurethane, the soft segment raw material is at least one of polydimethylsiloxane and polyhexamethylene oxide.

Optionally, in the molecular chain structure of polyurethane, the soft segment raw material includes polydimethylsiloxane and polyhexamethylene oxide.

Optionally, in the molecular chain structure of the polyurethane, the soft segment raw material includes polydimethylsiloxane and polyhexamethylene oxide, and the mass ratio of polydimethylsiloxane to polyhexamethylene oxide is 3:1 to 6:1.

Optionally, the hard segment content of the polyurethane is 30%-60%. Preferably, the hard segment content of the polyurethane is 35% to 60%.

Optionally, the isocyanate index of the polyurethane is 1.0 to 1.1. Preferably, the isocyanate index of the polyurethane is 1.0 to 1.05. Preferably, the isocyanate index of the polyurethane is 1.0 to 1.02.

Optionally, in Step 2, a solution of isocyanate containing a catalyst is applied to the film surface, and the application method includes at least one of immersion, coating, and spraying.

Optionally, in Step 2, a solution of isocyanate containing a catalyst is applied to the film surface, allowing the isocyanate to penetrate 5 to 30 μm from the film surface, followed by heating to initiate side-chain crosslinking.

Optionally, in Step 2, the isocyanate in the isocyanate solution is one of HMDI, MDI, and HDI, and the solvent is at least one of the following: cyclohexane, toluene, ethyl acetate, and cyclohexanone.

Optionally, in Step 2, the concentration of the isocyanate solution is 5 wt. % to 20 wt. %.

The present disclosure provides a method for preparing a prosthetic valve leaflet material, including:
Step 1, preparing a film with a thickness of 80 to 210 μm using a polyurethane with an elastic modulus of 5-30 MPa.
Step 2: applying an isocyanate solution containing a catalyst onto the surface of the film and reacting at 50-90° C. for 1-3 h; and
Step 3: placing the product of Step 2 in a modifier solution to react for 12-24 h at 30-50° C. to obtain the prosthetic valve leaflet material.

Several optional methods are further provided below, but they are not intended to be additional limitations on the above-mentioned overall solution. They are merely further supplements or optimizations. Under the premise that there are no technical or logical contradictions, each optional method can be combined separately for the above-mentioned overall solution, or multiple optional methods can be combined.

Optionally, the molecular chain structure of the polyurethane includes hard and soft segments, wherein the hard segments include first chain units formed from isocyanate and second chain units formed from a chain extender.

The isocyanate is at least one of the following: toluene diisocyanate (TDI), hexamethylene diisocyanate (HDI), diphenylmethane diisocyanate (MDI), naphthalene diisocyanate (NDI), p-phenylene diisocyanate (PPDI), isophorone diisocyanate (IPDI), xylylene diisocyanate (XDI), and triphenylmethane triisocyanate.

The chain extender is at least one of the following: ethylene glycol, propylene glycol, butanediol, pentanediol, hexanediol, octanediol, ethylenediamine, propylenediamine, butanediamine, pentanediamine, hexanediamine, octanediamine, and 1,3-bis(4-hydroxybutyl)-1,1,3,3-tetramethyldisiloxane (BHTD).

The soft segment raw material is at least one of the following: Dutral T5651, Dutral T5652, polyhexamethylene oxide (PHMO), polytetramethylene glycol (PTMO), adipic acid polyester diol, succinic acid polyester diol and polydimethylsiloxane diol.

Optionally, in the molecular chain structure of the polyurethane, the isocyanate is one of diphenylmethane diisocyanate, toluene diisocyanate, and hexamethylene diisocyanate.

Optionally, in the molecular chain structure of the polyurethane, the isocyanate is diphenylmethane diisocyanate.

Optionally, in the molecular chain structure of the polyurethane, the chain extender is at least one of ethylenediamine and 1,3-bis(4-hydroxybutyl)-1,1,3,3-tetramethyldisiloxane.

Optionally, in the molecular chain structure of the polyurethane, the chain extender includes ethylenediamine and 1,3-bis(4-hydroxybutyl)-1,1,3,3-tetramethyldisiloxane.

Optionally, in the molecular chain structure of the polyurethane, the chain extender includes ethylenediamine and 1,3-bis(4-hydroxybutyl)-1,1,3,3-tetramethyldisiloxane, and the molar ratio of ethylenediamine to 1,3-bis(4-hydroxybutyl)-1,1,3,3-tetramethyldisiloxane is 1:0.9 to 1:1.1.

Optionally, in the molecular chain structure of the polyurethane, the soft segment raw material is at least one of polydimethylsiloxane and polyhexamethylene oxide.

Optionally, in the molecular chain structure of polyurethane, the soft segment raw material includes polydimethylsiloxane and polyhexamethylene oxide.

Optionally, in the molecular chain structure of the polyurethane, the soft segment raw material includes polydimethylsiloxane and polyhexamethylene oxide, and the mass ratio of polydimethylsiloxane to polyhexamethylene oxide is 3:1 to 6:1.

Optionally, the hard segment content of the polyurethane is 30%-60%. Preferably, the hard segment content of the polyurethane is 35% to 60%.

Optionally, the isocyanate index of the polyurethane is 1.0 to 1.1. Preferably, the isocyanate index of the polyurethane is 1.0 to 1.05. Preferably, the isocyanate index of the polyurethane is 1.0 to 1.02.

Optionally, in Step 2, the isocyanate in the isocyanate solution is one of HMDI, MDI, and HDI, and the solvent is one of the following: cyclohexane, toluene, ethyl acetate, and cyclohexanone.

Optionally, in Step 2, the concentration of the isocyanate solution is 5 wt. % to 20 wt. %.

Optionally, the modifier in Step 3 is one of the following: 3-aminopropyltriethoxysilane, fluorine-containing alcohol, sphingosine phosphorylcholine, and 3-((tert-butyldimethylsilyl)oxy)-propanol.

Optionally, the solvent of the modifier in Step 3 is at least one of the following: cyclohexane, toluene, DMF, DMAc, and DMSO.

The present disclosure provides a method for preparing a valve leaflet, including:

Step 1: immersing a mold in the first surface-layer polyurethane solution, maintaining it for a predetermined time before removal, and drying it at 30-80° C. under an inert gas atmosphere to form a film.

The above Step 1 is repeated 1 to 9 times to form a first surface layer of the prosthetic valve leaflet material.

Step 2: immersing the mold in the inner-layer polyurethane solution, maintaining it for a predetermined time before removal, and drying it at 30-80° C. under an inert gas atmosphere to form a film.

The above Step 2 is repeated 1 to 9 times to form an inner layer of the prosthetic valve leaflet material.

Step 3: cutting into a leaflet shape and fitting the stent onto the mold.

Step 4, immersing the assembly in a second surface-layer polyurethane solution, maintaining it for a predetermined time before removal, and drying it at 30-80° C. under an inert gas atmosphere to form a film.

The above Step 4 is repeated 1 to 9 times to form a second surface layer of the prosthetic valve leaflet material.

Step 5: performing demolding by immersing the mold in water to obtain the valve leaflet.

The present disclosure provides a method for preparing a valve, including:

Step 1: immersing a mold in a first surface-layer polyurethane solution, maintaining it for a predetermined time before removal, and drying it at 30-80° C. under an inert gas atmosphere to form a film.

The above Step 1 is repeated 1 to 9 times to form a first surface layer of the prosthetic valve leaflet material.

Step 2: immersing the mold in an inner-layer polyurethane solution, maintaining it for a predetermined time before removal, and drying it at 30-80° C. under an inert gas atmosphere to form a film.

The above Step 2 is repeated 1 to 9 times to form the inner layer of the prosthetic valve leaflet material.

Step 3: cutting into a leaflet shape and fitting the stent onto the mold.

Step 4, immersing the assembly in a second surface-layer polyurethane solution, maintaining it for a predetermined time before removal, and drying it at 30-80° C. under an inert gas atmosphere to form a film.

The above Step 4 is repeated 1 to 9 times to form a second surface layer of the prosthetic valve leaflet material.

Step 5: performing demolding by immersing the mold in water to obtain the valve.

In the present disclosure, by adjusting the respective hard segment content or the type of chain extender in the surface-layer polyurethane and the inner-layer polyurethane, or by modifying or crosslinking the surface of the surface-layer polyurethane, the elastic modulus of the surface-layer polyurethane is increased compared to that of the inner-layer polyurethane. This enhances the intermolecular forces within the valve leaflet's surface material, strengthens the bonding between molecular chains, and densifies the material. As a result, the surface layer of the valve leaflet is less prone to defects, and the overall stability of the valve leaflet is improved. Through the design and modification of the surface-layer and inner-layer polyurethane materials, the preparation of a multifunctional biomimetic valve leaflet with a multilayered composite structure is achieved.

Compared with the prior art, the detailed mechanisms are as follows:

1) Compared with the relatively soft inner-layer polyurethane material, the increased hard segment content of the outer-layer polyurethane material reduces the content of soft segments susceptible to oxidative-hydrolytic attack, thereby enhancing intrinsic stability. The enhanced forces exerted by hard segments on the soft segments within the unit reduce the mobility of soft segments, making it difficult for water and oxygen to transport through the surface layer into the interior. This protects the softer inner-layer polyurethane from oxidation. Restricted movement of soft segments also reduces the likelihood of relative displacement among soft segments, thereby improving creep resistance. This achieves a multifunctional biomimetic valve leaflet with a multilayered composite structure, featuring a soft inner layer (having a high soft segment content in the inner-layer polyurethane material) and highly durable surface layers (having a high hard segment content in the surface-layer polyurethane material).

2) The surface-layer polyurethane of the valve leaflet employs amine as the chain extender. The reaction between amine and isocyanate generates urea linkages, which form twice as many hydrogen bonds with soft segments as urethane linkages do. This significantly enhances the interaction forces between hard segments and soft segments, resulting in a stronger surface-layer polyurethane material. Simultaneously, the reduced mobility of soft segments in the molecular structure impedes the transport of water and oxygen through the surface layer into the interior, protecting the inner-layer polyurethane from oxidation. Compared with the inner material, the use of amine-based chain extender in the surface-layer material restricts the movement of soft segments through urea linkages, reducing the possibility of relative displacement among soft segments and effectively improving the creep resistance of the surface-layer valve leaflet. Consequently, the protection of the inner layer is enhanced, achieving the preparation of a biomimetic leaflet with a soft inner layer and a protective outer layer, thereby realizing a multilayer composite multifunctional biomimetic valve leaflet.

3) Crosslinking the surface-layer polyurethane material of the valve leaflet greatly increases the interaction forces between molecular chains in the surface layer, enhancing the strength of the leaflet surface. This reduces the likelihood of hydrolysis in the inner-layer polyurethane material, which has higher softness, due to water and oxygen intrusion. The increased interaction forces between molecular chains in the surface-layer polyurethane also limit creep, resulting in an integrated polyurethane leaflet design where the surface layer achieves effective protective tolerance and the inner layer maintains soft and flexible leaflet motion. This imparts improved biostability, biocompatibility, creep resistance, and fatigue resistance to the prosthetic valve leaflet material, achieving the fabrication of a multifunctional biomimetic valve leaflet with a multilayered composite structure.

4) By introducing isocyanate into the surface-layer polyurethane material of the valve leaflet for modification, side chains are effectively grafted to the main chain, increasing steric hindrance between segments. The presence of side chains reduces mobility between chains, enhancing the tolerance of the surface-layer polyurethane material. This decreases the transmission of water and oxygen inside the polyurethane and reduces creep. Simultaneously, the side chains tend to distribute on the surface, contributing to an integrated polyurethane leaflet design that enables soft and flexible leaflet motion. This configuration additionally provides further protection to the internal structure, improves overall biostability, and ultimately facilitates the fabrication of a multifunctional biomimetic valve leaflet with a multilayered composite structure.

### BENEFICIAL EFFECTS

The synthetic valve leaflet material provided in this disclosure enhances the tolerance of the surface layer of the valve leaflet by adjusting the hard segment content of the surface-layer polyurethane, selecting the type of chain extender, performing graft modification, or introducing crosslinking. Meanwhile, the inner-layer polyurethane remains relatively soft to ensure effective flexible motion. Given the relatively small proportion of the surface layer to the overall thickness of the valve leaflet, it maintains a balance among hemodynamic performance, biostability, biocompatibility, creep resistance, and fatigue resistance, thereby achieving the performance required for heart valve leaflets. This allows the preparation of a multifunctional biomimetic valve leaflet with a multilayered composite structure.

### BRIEF DESCRIPTION OF DRAWINGS

FIG. 1 is an SEM image of the valve leaflet material prepared in Example 1 after oxidation-resistance testing.

FIG. 2 is an SEM image of the valve leaflet material prepared in Example 2 after oxidation-resistance testing.

FIG. 3 is an SEM image of the valve leaflet material prepared in Example 3 after oxidation-resistance testing.

FIG. 4 is an SEM image of the valve leaflet material prepared in Example 4 after oxidation-resistance testing.

FIG. 5 is an SEM image of the valve leaflet material prepared in Example 5 after oxidation-resistance testing.

FIG. 6 is an SEM image of the valve leaflet material prepared in Example 6 after oxidation-resistance testing.

FIG. 7 is an SEM image of the valve leaflet material prepared in Comparative Example 1 after oxidation-resistance testing.

FIG. 8 is an SEM image of the valve leaflet material prepared in Comparative Example 2 after oxidation-resistance testing.

FIG. 9 is a schematic view of the stent being fitted onto the mold.

FIG. 10 is a schematic structural view of the valve leaflet material.

### DESCRIPTION OF EMBODIMENTS

Technical solutions of embodiments of the present disclosure will be clearly and completely described below in combination with the drawings. Obviously, the described embodiments are only part of, rather than all of the embodiments of the present disclosure. Based on the embodiments of this disclosure, all other embodiments obtained by those ordinary skilled in the art without any creative work shall fall within the protection scope of this disclosure.

In order to better describe and illustrate the embodiments of the present disclosure, reference may be made to one or more figures, but the additional details or examples used to describe the figures should not be construed as limiting the scope of the disclosure, the embodiments currently described, or the preferred embodiments of the present disclosure.

It should be noted that when a component is referred to as “connected” to another component, it may be directly connected to another component or there may be a middle component connected therebetween. When a component is considered to be “fitted onto” another component, it may be directly fitted onto another component or there may be a middle component arranged therebetween.

Unless defined otherwise, all technical and scientific terms used herein have the same meaning as commonly understood by one of ordinary skill in the art to which this disclosure belongs. The terms used in the specification of the present disclosure are only for the purpose of describing specific embodiments and are not intended to limit the present disclosure.

As shown in FIG. 10, a prosthetic valve leaflet material made of polyurethane includes: an inner layer 200, and a surface layer 100 located on two opposite sides of the inner layer in the thickness direction, wherein the elastic modulus of the surface-layer polyurethane is greater than that of the inner-layer polyurethane.

The prosthetic valve leaflet material provided in the present disclosure adopts a multilayered composite structure. The polyurethane located in the surface has a larger elastic modulus, while the polyurethane located in the inner layer has a smaller elastic modulus. The performance of the surface-layer polyurethane and the inner-layer polyurethane complement each other, mimicking the structural design of natural leaflet valves. The inner layer with a smaller elastic modulus ensures the flexibility of the valve leaflet and guarantees its normal opening and closing. The surface-layer polyurethane offers more stable biocompatibility and biostability. Moreover, since the thickness of the surface-layer polyurethane is smaller than the overall thickness, it has minimal effect on the overall softness. Additionally, the low water and oxygen permeability of the surface-layer polyurethane effectively protects the inner-layer polyurethane. Therefore, the polyurethane valve with the multilayered composite structure can take into account fluid mechanics, biostability, biocompatibility, creep resistance and fatigue resistance simultaneously.

The biostability, fatigue resistance, biocompatibility, and creep resistance of the material are related to its elastic modulus. Macroscopically, the elastic modulus characterizes the ease with which a material undergoes elastic deformation under a certain stress, and a higher elastic modulus indicates greater resistance to deformation. From the perspective of interatomic forces, the elastic modulus characterizes the strength of the material's internal bonding. Therefore, a higher elastic modulus corresponds to greater internal bonding of the polyurethane, which restricts the mobility of soft segments and reduces the transmission of water and oxygen within the polyurethane. This results in less internal exposure to water and oxygen, thereby enhancing the material's biostability. Furthermore, stronger interaction forces help confine small molecules within the polymer, reducing the release of small molecules that could induce immune responses, thus resulting in better biocompatibility. The irreversible creep primarily arises from the displacement between molecular chains. The stronger the interaction forces, the more difficult it becomes for molecular chains to displace, thereby significantly enhancing the creep resistance. As can be seen from the above, a higher elastic modulus significantly leads to notable improvements in biostability, fatigue resistance, biocompatibility, and creep resistance. For example, in the design of this solution, since the surface-layer polyurethane directly contacts blood, the use of polyurethane with a higher elastic modulus can simultaneously meet the requirements for biostability, fatigue resistance, biocompatibility, and creep resistance. Meanwhile, the inner-layer polyurethane with a lower elastic modulus enhances the flexibility (as biological valve leaflets are inherently soft). This ensures the valve leaflet possesses favorable hydrodynamic performance, thereby guaranteeing the overall hydrodynamic performance of the multilayered prosthetic valve leaflet material.

The creep in material is primarily caused by three types of deformation: instantaneous elastic deformation, hyperelastic deformation, and viscous flow. Instantaneous elastic deformation originates from changes in bond angles and bond lengths. It has a relatively small deformation magnitude, but the deformation recovers immediately once the external force is removed. Hyperelastic deformation results from the gradual extension of molecular chains through segmental motion. Its deformation magnitude is much larger than that of instantaneous elastic deformation, and the deformation exhibits an exponential relationship with time. After the external force is removed, the hyperelastic deformation can recover slowly. Viscous flow arises from the relative movement between molecular chains, and this process is irreversible. The interaction forces within the surface-layer polyurethane are enhanced due to the higher hard segment content, the use of the amine-containing chain extender, or modification and crosslinking of the surface-layer polyurethane. This makes the relative movement between molecular chains more difficult, reduces the ability of viscous flow, and thereby improves the creep resistance of the product.

Additionally, as the valve leaflet moves in sync with the heartbeat and undergoes periodic bending, the prepared prosthetic valve leaflet material is required to exhibit excellent bending resistance. The design of this solution enables the outer layer of the prosthetic valve leaflet material to undergo greater bending deformation relative to the inner layer when subjected to bending. The surface layer utilizes polyurethane with a higher elastic modulus, specifically a harder polyurethane, which offers greater resistance to bending. This enhances the overall bending resistance of the prosthetic valve leaflet material.

The elastic modulus of the surface-layer polyurethane is 10 to 60 MPa greater than that of the inner-layer polyurethane, preferably 10 to 30 MPa.

The elastic modulus of the surface-layer polyurethane is 30-100 MPa, preferably 40-60 MPa. The elastic modulus of the inner-layer polyurethane is 5-30 MPa, preferably 20-30 MPa.

The oxygen permeability of the surface-layer polyurethane is ≤9*10⁹ (cm²/sec*cm*Hg).

The inner-layer polyurethane does not directly contact the implantation environment. Consequently, the oxygen amount to which the inner-layer polyurethane is exposed depends primarily on the permeability of the surface-layer polyurethane. The lower the permeability of the surface-layer polyurethane, the lower the content of water and oxygen reaching the inner layer, thereby reducing its susceptibility to hydrolysis and oxidation. The transport of substances within polyurethane is mainly through the soft segments. Substances reside in the spaces of the soft segments and are transported from one side to the other as the soft segments move. Therefore, the permeability of polyurethane depends on both the content of the soft segment and the mobility of the soft segments, as well as the nature of the transported substance. Generally, higher affinity and better compatibility between the substance and the polymer lead to greater transmission and higher permeability. In this context, the transported substances are defined as water (which induces hydrolysis) and/or oxygen (which induces oxidation). Thus, to reduce the permeability of the surface-layer polyurethane, hydrophobic soft segments, a lower soft segment content, and polyurethanes with stronger interaction forces are primarily selected. To simultaneously ensure biocompatibility, the choice of soft segments for the surface-layer polyurethane is constrained. Therefore, strategies focus more on reducing the hard segment content in the surface-layer polyurethane or enhancing the interaction forces between the soft and hard segments. This can be achieved, for example, by increasing urea linkages, introducing crosslinking, or leveraging the chain-pinning effect of branched structures.

Based on the design requirements for the overall performance of the valve leaflet, the molecular chain structure of the surface-layer polyurethane and inner-layer polyurethane used to prepare the valve leaflet material should meet at least one of the following conditions:
a. the hard segment content of the inner-layer polyurethane is lower than that of the surface-layer polyurethane;
b. the inner-layer polyurethane is prepared using an alcohol-based chain extender, and the surface-layer polyurethane is prepared using an amine-containing chain extender; and
c. the surface-layer polyurethane has a higher crosslink density compared with the inner-layer polyurethane.

In condition b, the amine-containing chain extender may be either a single amine-based chain extender or a mixture of amine-based and alcohol-based chain extenders.

In condition c, the difference between the surface-layer polyurethane and the inner-layer polyurethane lies in the crosslink density. This includes scenarios where the surface-layer polyurethane is crosslinked while the inner-layer polyurethane is not, as well as scenarios where both layers are crosslinked but the surface layer has a higher crosslink density. Apart from the difference in crosslinking, there are no significant variations in other molecular structures between the two layers.

The three conditions a, b, and c may be met simultaneously, or only two or even just one of them may be met. The fulfillment of any single condition a, b, or c can enable the surface-layer and inner-layer polyurethanes to exhibit different elastic modulus.

The design of the surface-layer polyurethane material provides biostability and biocompatibility for the valve leaflet material. Meanwhile, the strong interaction forces between the internal soft and hard segments restrict the movement of the soft segment, reduce its water and oxygen transmission capacity, thereby protecting the inner-layer polyurethane and enhancing the overall biostability of the valve leaflet. The inner-layer polyurethane leaflet material is designed to achieve the hydrodynamic performance of the valve leaflet. Since the inner-layer polyurethane constitutes a larger proportion in the thickness direction, it directly determines the mechanical properties of the valve leaflet.

The molecular chain structure of the surface-layer polyurethane includes hard and soft segments, wherein the hard segments include first chain units formed from isocyanate and second chain units formed from a chain extender.

The isocyanate is at least one of the following: toluene diisocyanate (TDI), hexamethylene diisocyanate (HDI), diphenylmethane diisocyanate (MDI), naphthalene diisocyanate (NDI), p-phenylene diisocyanate (PPDI), isophorone diisocyanate (IPDI), xylylene diisocyanate (XDI), and triphenylmethane triisocyanate.

The chain extender is at least one of the following: ethylene glycol, propylene glycol, butanediol, pentanediol, hexanediol, octanediol, ethylenediamine, propylenediamine, butanediamine, pentanediamine, hexanediamine, octanediamine, and 1,3-bis(4-hydroxybutyl)-1,1,3,3-tetramethyldisiloxane (BHTD).

The soft segment raw material is at least one of the following: Dutral T5651, Dutral T5652, polyhexamethylene oxide (PHMO), polytetramethylene glycol (PTMO), adipic acid polyester diol, succinic acid polyester diol, and polydimethylsiloxane diol.

In the molecular chain structure of the surface-layer polyurethane, the isocyanate is one of diphenylmethane diisocyanate, toluene diisocyanate and hexamethylene diisocyanate.

In the molecular chain structure of the surface-layer polyurethane, the isocyanate is diphenylmethane diisocyanate.

In the molecular chain structure of the surface-layer polyurethane, the chain extender is at least one of ethylenediamine and 1,3-bis(4-hydroxybutyl)-1,1,3,3-tetramethyldisiloxane.

In the molecular chain structure of the surface-layer polyurethane, the chain extender includes ethylenediamine and 1,3-bis(4-hydroxybutyl)-1,1,3,3-tetramethyldisiloxane.

In the molecular chain structure of the surface-layer polyurethane, the chain extender includes ethylenediamine and 1,3-bis(4-hydroxybutyl)-1,1,3,3-tetramethyldisiloxane, and the molar ratio of ethylenediamine to 1,3-bis(4-hydroxybutyl)-1,1,3,3-tetramethyldisiloxane is 1:0.9 to 1.1.

In the molecular chain structure of the surface-layer polyurethane, the soft segment raw material is at least one of polydimethylsiloxane and polyhexamethylene oxide.

In the molecular chain structure of the surface-layer polyurethane, the soft segment raw material includes polydimethylsiloxane and polyhexamethylene oxide.

In the molecular chain structure of the surface-layer polyurethane, the soft segment raw material includes polydimethylsiloxane and polyhexamethylene oxide, and the mass ratio of polydimethylsiloxane to polyhexamethylene oxide is 3:1 to 6:1.

The molecular chain structure of the inner-layer polyurethane includes hard and soft segments, wherein the hard segments include first chain units formed from isocyanate and second chain units formed from a chain extender.

The isocyanate is at least one of the following: toluene diisocyanate (TDI), hexamethylene diisocyanate (HDI), diphenylmethane diisocyanate (MDI), naphthalene diisocyanate (NDI), p-phenylene diisocyanate (PPDI), isophorone diisocyanate (IPDI), xylylene diisocyanate (XDI), and triphenylmethane triisocyanate.

The chain extender is at least one of the following: ethylene glycol, propylene glycol, butanediol, pentanediol, hexanediol, octanediol, ethylenediamine, propylenediamine, butanediamine, pentanediamine, hexanediamine, octanediamine, and 1,3-bis(4-hydroxybutyl)-1,1,3,3-tetramethyldisiloxane (BHTD).

The soft segment raw material is at least one of the following: Dutral T5651, Dutral T5652, polyhexamethylene oxide (PHMO), polytetramethylene glycol (PTMO), adipic acid polyester diol, succinic acid polyester diol and polydimethylsiloxane diol.

In the molecular chain structure of the inner-layer polyurethane, the isocyanate is one of diphenylmethane diisocyanate, toluene diisocyanate and hexamethylene diisocyanate.

In the molecular chain structure of the inner-layer polyurethane, the isocyanate is diphenylmethane diisocyanate.

In the molecular chain structure of the inner-layer polyurethane, the chain extender is at least one of ethylenediamine and 1,3-bis(4-hydroxybutyl)-1,1,3,3-tetramethyldisiloxane.

In the molecular chain structure of the inner-layer polyurethane, the chain extender includes ethylenediamine and 1,3-bis(4-hydroxybutyl)-1,1,3,3-tetramethyldisiloxane.

In the molecular chain structure of the inner-layer polyurethane, the chain extender includes ethylenediamine and 1,3-bis(4-hydroxybutyl)-1,1,3,3-tetramethyldisiloxane, and the molar ratio of ethylenediamine to 1,3-bis(4-hydroxybutyl)-1,1,3,3-tetramethyldisiloxane is 1:0.9 to 1:1.1.

In the molecular chain structure of the inner-layer polyurethane, the soft segment raw material is at least one of polydimethylsiloxane and polyhexamethylene oxide.

In the molecular chain structure of the inner-layer polyurethane, the soft segment raw material includes polydimethylsiloxane and polyhexamethylene oxide.

In the molecular chain structure of the inner-layer polyurethane, the soft segment raw material includes polydimethylsiloxane and polyhexamethylene oxide, and the mass ratio of polydimethylsiloxane to polyhexamethylene oxide is 3:1 to 6:1.

The hard segment content of the surface-layer polyurethane and the inner-layer polyurethane is 30% to 60%.

The hard segment content of the surface-layer polyurethane and the inner-layer polyurethane is 35% to 60%.

The isocyanate index of the surface-layer polyurethane and the inner-layer polyurethane is 1.0 to 1.1.

The isocyanate index of the surface-layer polyurethane and the inner-layer polyurethane is 1.0 to 1.05.

The isocyanate index of the surface-layer polyurethane and the inner-layer polyurethane is 1.0 to 1.02.

The thickness of the prosthetic valve leaflet material is 75 to 210 μm.

The thickness of the prosthetic valve leaflet material is 80 to 210 μm, preferably 130 to 200 μm.

The thickness of the inner-layer polyurethane is 70 to 170 μm, and the thickness of the surface-layer polyurethane is 5 to 40 μm.

The thickness of the inner-layer polyurethane is 70 to 170 μm, and the thickness of the surface-layer polyurethane is 10 to 40 μm.

The thickness of the inner-layer polyurethane is 70 to 150 μm, and the thickness of the surface-layer polyurethane is 5 to 30 μm.

The two opposite sides of the inner-layer polyurethane are respectively provided with surface-layer polyurethane, and the thickness of the surface-layer polyurethane refers to the thickness of the surface-layer polyurethane on one side of the inner-layer polyurethane, rather than the sum of the thickness of the surface-layer polyurethane on both sides of the inner-layer polyurethane.

The thickness of the surface-layer polyurethane needs to meet at least two requirements. On the one hand, it should not be so thick as to adversely affect the overall flexibility of the prosthetic valve leaflet material. On the other hand, it should not be so thin as to compromise its effective barrier against blood oxygen in the blood environment, thereby failing to provide adequate protection for the inner-layer polyurethane.

The appropriate thickness distribution of the inner-layer polyurethane and the surface-layer polyurethane enables the prosthetic valve leaflet material to have good fluid mechanical properties as a whole, as well as to meet the requirements of biostability, biocompatibility, creep resistance and fatigue resistance.

The inner layer includes at least one first sub-layer along the thickness direction, each first sub-layer being made of the same type of polyurethane, or the elastic modulus of each first sub-layer gradually increases by a predetermined interval from the inside to the outside in the thickness direction. As shown in FIG. 10, when referring to “from the inside to the outside”, it means, in the thickness direction, starting from the central line position and extending in the direction indicated by the arrow, which is defined as “from the inside to the outside”.

In the thickness direction, the direction extending from the center of the prosthetic valve leaflet material to the outside, that is, from the inside to the outside in the thickness direction.

The elastic modulus of each first sub-layer increases gradually at an interval of 5 to 10 MPa.

The inner layer includes 1 to 6 first sub-layers, with each first sub-layer divided into at least two groups. First sub-layers within the same group share identical elastic modulus, and the elastic modulus of first sub-layers within each group gradually increases at a predetermined interval from the inside to the outside in the thickness direction.

The surface layer includes at least one second sub-layer along the thickness direction, and each second sub-layer is made of the same type of polyurethane, or the elastic modulus of each second sub-layer gradually increases by a predetermined interval from the inside to the outside in the thickness direction.

The elastic modulus of each second sub-layer increases gradually at an interval of 5 to 10 MPa.

The surface layer includes 2 to 4 second sub-layers, each second sub-layer is divided into at least two groups, the elastic modulus of each second sub-layer in the same group is the same, and the elastic modulus of the each group of second sub-layers gradually increases from the inside to the outside in the thickness direction at intervals of predetermined values.

The first sub-layer and the second sub-layer are layer structures formed during the manufacturing process based on specific procedures. For example, a first sub-layer or a second sub-layer is formed by performing one cycle of immersion and drying.

The hard segment content of the inner-layer polyurethane is lower than that of the surface-layer polyurethane.

The surface-layer polyurethane exhibits a relatively higher elastic modulus, which can be achieved by increasing the hard segment content. The relatively higher hard segment content enhances interaction forces between the molecular chains, resulting in a denser material structure that is less prone to defects. Consequently, the area of calcification deposition is reduced, and the biocompatibility is relatively improved. Concurrently, the enhanced interaction forces between the molecular chains contribute to better biostability, fatigue resistance, and creep resistance of the material.

Meanwhile, a higher hard segment content also implies a lower soft segment content. Consequently, issues related to oxidation and hydrolysis, which are inherent to the soft segment structure, are correspondingly reduced, and the biostability, fatigue resistance, creep resistance and biocompatibility of the material are significantly improved.

The inner-layer-polyurethane, with a lower hard segment content, possesses greater flexibility. Under blood flow conditions, it demonstrates superior hemodynamic performance. The inner-layer polyurethane and the surface-layer polyurethane cooperate with each other, achieving a balance between hemodynamic performance, biostability, biocompatibility, creep resistance, and fatigue resistance. fluid mechanics, biostability, biocompatibility, creep resistance and fatigue resistance.

For polyurethane materials, higher water and oxygen content will cause oxidation and hydrolysis of the soft segment. To mitigate these issues, it is essential to reduce the internal water and oxygen content within the prosthetic valve leaflet material as much as possible. In terms of usage environment, the surface-layer polyurethane is exposed to significantly higher levels of water and oxygen compared to the inner layer. With higher hard segment content and stronger interaction forces between the molecular chains, the surface-layer polyurethane provides more effective resistance against the penetration of water and oxygen. Consequently, even if the inner-layer polyurethane may have a relatively higher soft segment content, its reduced exposure to water and oxygen substantially diminishes the associated oxidation and hydrolysis issues.

The hard segment content of the surface-layer polyurethane is 45% to 60%, and the hard segment content of the inner-layer polyurethane is 30% to 45%.

The hard segments in the molecular chain structure of the surface-layer polyurethane and the inner-layer polyurethane are modified in one of the following ways:
A. Introducing fluorine-containing side chains; and
B. Introduce a chain extender containing POSS groups.

The introduction of fluorine-containing side chains is achieved by:
adding a fluorine-containing diisocyanate with a mass fraction of 3% to 10% to the synthetic raw materials of the polyurethane.

The fluorine-containing diisocyanate is prepared using existing technology, for example, by reacting a triisocyanate with a fluorine-containing alcohol (such as 4-fluorobenzyl alcohol, CAS No.: 459-56-3, or 1H, 1H-perfluorododecane-1-ol, CAS No.: 423-65-4).

The fluorine-containing side chains can induce a pinning effect, making it difficult for the soft segments of polyurethane to move, thereby enhancing the hardness and strength of polyurethane. Meanwhile, fluorine-containing side chains can also bring about a steric effect, tending to migrate to the surface of polyurethane. Due to the excellent hydrophobicity of the fluorine-containing groups, water penetration into the polyurethane matrix is significantly impeded, thereby improving the material's resistance to hydrolysis.

The introduction of POSS groups is achieved by:
adding a POSS-containing diol chain extender with a mass fraction of 1% to 3% to the synthetic raw materials of the polyurethane.

The (diol) chain extender containing POSS groups may be trans-cyclohexanediol heptyl-cage polysilsesquioxane, CAS number: 408439-48-3.

A small amount of POSS groups functions similarly to incorporating inorganic fillers into polymer materials, which significantly enhances the strength and wear resistance of polyurethane, while also improving the hardness. Due to the steric hindrance effect, POSS tends to be arranged on the surface of polyurethane, and the siloxane in POSS provides polyurethane with excellent biostability and biocompatibility.

Modifying the hard segments in the molecular chain structure of the surface-layer polyurethane and the inner-layer polyurethane can increase the elastic modulus of the polyurethane while also improving its biostability and biocompatibility.

The inner-layer polyurethane is prepared by using an alcohol-based chain extender, and the surface-layer polyurethane is prepared by using an amine-based chain extender, which may be a single amine-based chain extender or a mixture of amine-containing and alcohol-based chain extenders.

The alcohol-based chain extender is at least one of the following: ethylene glycol, propylene glycol, butanediol, pentanediol, hexanediol, octanediol, and 1,3-bis(4-hydroxybutyl)-1,1,3,3-tetramethyldisiloxane (BHTD).

The amine-based chain extender is at least one of the following: ethylenediamine, propylenediamine, butylenediamine, pentamethylenediamine, hexamethylenediamine, and octanediamine.

The alcohol-based chain extender is butanediol.

The amine-based chain extender is ethylenediamine.

The alcohol-based chain extender is butanediol, and the amine-based chain extender is ethylenediamine.

Polyurethanes prepared with different chain extenders exhibit varying interaction forces between the soft and hard segments, attributed to the differences in the chain extenders. Specifically, when an amine-based chain extender is used, urea linkages are generated in the hard segments, with each urea linkage forming two hydrogen bonds with the soft segments; and when an alcohol-based chain extender is used, urethane linkages are generated in the hard segments, with each urethane linkage forming only one hydrogen bond with the soft segments. Consequently, urea linkages provide significantly stronger interaction forces than urethane linkages. Polyurethanes with stronger interaction forces exhibit higher elastic moduli, while those with weaker interaction forces exhibit lower elastic moduli. The polyurethane with a higher elastic modulus serves as the surface layer, while the polyurethane with a lower elastic modulus serves as the inner layer. The strong interaction forces between the soft and hard segments within the surface-layer polyurethane result in a denser structure that resists penetration by external water and oxygen, thereby protecting the inner-layer polyurethane. Furthermore, the higher interaction forces between the molecular chains in the surface-layer polyurethane also restrict the overall creep of the valve leaflet, improving the overall creep resistance of the valve.

Meanwhile, when the prosthetic valve leaflet bends, the bending degree of the surface is greater than that of the interior. Consequently, cracking usually initiates at the surface. The surface-layer polyurethane with intermolecular chain interactions can delay crack initiation and enhance the fatigue resistance of the valve leaflet.

Since the thickness of the surface-layer polyurethane is relatively thin, it has little impact on the overall mechanical properties of the film. Therefore, combining the surface-layer polyurethane and the inner-layer polyurethane enables the prosthetic valve leaflet material to achieve superior biostability, biocompatibility, creep resistance, and fatigue resistance without compromising its own fluid properties.

The surface-layer polyurethane undergoes crosslinking modification.

The inner-layer polyurethane is free of crosslinking modification, whereas only the surface-layer polyurethane undergoes crosslinking modification. This allows the layers to exhibit distinct yet complementary properties. Specifically, after crosslinking, the interaction forces between the molecular chains of the surface-layer polyurethane are greatly increased, reducing the probability of water and oxygen intrusion leading to hydrolysis of the inner-layer polyurethane. The increased interaction forces between the molecular chains of the surface-layer polyurethane also restrict the occurrence of creep, so that the prosthetic valve leaflet material has better biostability, biocompatibility, creep resistance, and fatigue resistance. Meanwhile, as the inner-layer polyurethane remains uncrosslinked, it retains favorable hydrodynamic performance. The complementary properties of the inner- and surface-layer polyurethane synergize to ultimately provide the prosthetic valve leaflet material with enhanced biostability, biocompatibility, creep resistance, fatigue resistance, and hydrodynamic performance.

The crosslinking modification of the surface-layer polyurethane is carried out by radiation crosslinking or chemical crosslinking.

A valve leaflet made of the prosthetic valve leaflet material.

A valve, including:
a stent, with a blood flow channel defined therein; and
one or more leaflets, wherein the valve leaflets are made of the prosthetic valve leaflet material.

A method for preparing a prosthetic valve leaflet material, including:

Step 1: immersing a mold in a first surface-layer polyurethane solution, maintaining the mold for a predetermined time before removal, and drying it at 30-80° C. under an inert gas atmosphere to form a film.

The above Step 1 is repeated 1 to 9 times to form a first surface layer of the prosthetic valve leaflet material.

Step 2: immersing the mold in an inner-layer polyurethane solution, maintaining it for a predetermined time before removal, and drying it at 30-80° C. under an inert gas atmosphere to form a film.

The above Step 2 is repeated 1 to 9 times to form an inner layer of the prosthetic valve leaflet material.

Step 3: fitting a stent onto the mold, immersing the assembly in a second surface-layer polyurethane solution, maintaining it for a predetermined time before removal, and drying it at 30-80° C. under an inert gas atmosphere to form a film.

The above Step 3 is repeated 1 to 9 times to form a second surface layer of the prosthetic valve leaflet material.

Step 4: performing demolding by immersing the mold in water to obtain the prosthetic valve leaflet material.

In Step 1, the first surface layer is formed by 1 to 9 times of repeated film formation, wherein each film formation corresponds to a sub-layer, and all sub-layers jointly constitute the first surface layer. The same principle applies to explain the processes in Steps 2 and 3.

The contact surface of each sub-layer is formed through solution-phase contact, permeation, and solidification at adjacent interfaces. Since the same material (all being polyurethane) is used, the compatibility between the sub-layers is improved, resulting in stronger interfacial bonding.

The first surface-layer polyurethane and the second surface-layer polyurethane may be the same or different, with both being categorized as surface-layer polyurethane.

The hard segment content of the surface-layer polyurethane is 45 to 60%, and the hard segment content of the inner-layer polyurethane is 30 to 45%.

The inner-layer polyurethane is prepared by using an alcohol-based chain extender, and the surface-layer polyurethane is prepared by using an amine-containing chain extender.

The alcohol-based chain extender is at least one of the following: ethylene glycol, propylene glycol, butanediol, pentanediol, hexanediol, octanediol, and 1,3-bis(4-hydroxybutyl)-1,1,3,3-tetramethyldisiloxane (BHTD).

The amine-based chain extender is at least one of the following: ethylenediamine, propylenediamine, butylenediamine, pentamethylenediamine, hexamethylenediamine, and octanediamine.

The concentration of the first surface-layer polyurethane solution is 10-30% (w/v). The concentration of the second surface-layer polyurethane solution is 5-15% (w/v).

The concentration of the inner-layer polyurethane solution is 10-30% (w/v).

The solvents for the first surface-layer polyurethane solution, the second surface-layer polyurethane solution, and the inner-layer polyurethane solution are each selected from one of the following: N,N-dimethylacetamide (DMAc), dimethylformamide (DMF), dimethyl sulfoxide (DMSO), toluene, and tetrahydrofuran (THF).

In Step 1, the drying time is 1 to 6 h.

In Step 2, the drying time is 1 to 6 h.

In Step 3, the drying time is 1 to 12 h.

In Step 1, the predetermined maintaining time in the first surface-layer polyurethane solution is 5 to 10 s.

In Step 2, the predetermined maintaining time in the inner-layer polyurethane solution is 5 to 10 s.

In Step 3, the predetermined maintaining time in the second surface-layer polyurethane solution is 5 to 10 s.

A method for preparing a prosthetic valve leaflet material, including:

Step 1, preparing a film with a thickness of 80 to 210 μm from polyurethane having an elastic modulus of 5 to 30 MPa; and

Step 2: applying an isocyanate solution containing a catalyst onto the surface of the film, and reacting at 50 to 90° C. for 3-5 h to obtain the prosthetic valve leaflet material.

The molecular chain structure of polyurethane includes hard and soft segments, wherein the hard segments include first chain units formed from isocyanate and second chain units formed from a chain extender.

The isocyanate is at least one of the following: toluene diisocyanate (TDI), hexamethylene diisocyanate (HDI), diphenylmethane diisocyanate (MDI), naphthalene diisocyanate (NDI), p-phenylene diisocyanate (PPDI), isophorone diisocyanate (IPDI), xylylene diisocyanate (XDI), and triphenylmethane triisocyanate.

The chain extender is at least one of the following: ethylene glycol, propylene glycol, butanediol, pentanediol, hexanediol, octanediol, ethylenediamine, propylenediamine, butanediamine, pentanediamine, hexanediamine, octanediamine, and 1,3-bis(4-hydroxybutyl)-1,1,3,3-tetramethyldisiloxane (BHTD).

The soft segment raw material is at least one of the following: Dutral T5651, Dutral T5652, polyhexamethylene oxide (PHMO), polytetramethylene glycol (PTMO), adipic acid polyester diol, succinic acid polyester diol and polydimethylsiloxane diol.

In the molecular chain structure of the polyurethane, the isocyanate is one of diphenylmethane diisocyanate, toluene diisocyanate and hexamethylene diisocyanate.

In the molecular chain structure of the polyurethane, the isocyanate is diphenylmethane diisocyanate.

In the molecular chain structure of the polyurethane, the chain extender is at least one of ethylenediamine and 1,3-bis(4-hydroxybutyl)-1,1,3,3-tetramethyldisiloxane.

In the molecular chain structure of the polyurethane, the chain extender includes ethylenediamine and 1,3-bis(4-hydroxybutyl)-1,1,3,3-tetramethyldisiloxane.

In the molecular chain structure of the polyurethane, the chain extender includes ethylenediamine and 1,3-bis(4-hydroxybutyl)-1,1,3,3-tetramethyldisiloxane, and the molar ratio of ethylenediamine to 1,3-bis(4-hydroxybutyl)-1,1,3,3-tetramethyldisiloxane is 1:0.9 to 1:1.1.

In the molecular chain structure of the polyurethane, the soft segment raw material is at least one of polydimethylsiloxane and polyhexamethylene oxide.

In the molecular chain structure of polyurethane, the soft segment raw material includes polydimethylsiloxane and polyhexamethylene oxide.

In the molecular chain structure of the polyurethane, the soft segment raw material includes polydimethylsiloxane and polyhexamethylene oxide, and the mass ratio of polydimethylsiloxane to polyhexamethylene oxide is 3:1 to 6:1.

The hard segment content of polyurethane is 30%-60%. The hard segment content of polyurethane is 35% to 60%.

The isocyanate index of polyurethane is 1.0-1.1. Preferably, the isocyanate index of polyurethane is 1.0-1.05. Preferably, the isocyanate index of polyurethane is 1.0-1.02.

The isocyanate solution containing a catalyst is applied to the surface of the film, where the isocyanate in the isocyanate solution reacts with the molecular chains of the polyurethane to form a crosslinked structure. Specifically, a single isocyanate molecule chemically bonds with the reactive sites of two polyurethane molecular chains, to form an interlinked structure between the polyurethane molecular chains. The reaction temperature and time for isocyanate side chain crosslinking are relatively high.

The film in Step 1 can be prepared by using existing techniques, such as dip coating, hot pressing, or other forming methods.

In Step 2, the isocyanate solution containing a catalyst is applied to the surface of the film, and the application method includes at least one of immersion, coating, and spraying.

In Step 2, the isocyanate solution containing a catalyst is applied to the surface of the film, the isocyanate is infiltrated within a range of 5 to 30 μm from the surface of the film, and the heating reaction initiates side chain crosslinking.

With in a range of 5 to 30 μm from the film surface, an isocyanate solution permeates and, under the action of a catalyst, initiates side-chain crosslinking. This forms, within a range of 5 to 30 μm from the film surface, a polyurethane with a different structure from the original structure of the film. This crosslinked polyurethane constitutes the surface-layer polyurethane, while the inner-layer polyurethane has the original film structure of the thin film inside the surface-layer polyurethane.

When the polyurethane penetrates into the film surface, it is necessary to control the penetration depth to be within a range of 5 to 30 μm from the film surface. Isocyanate infiltration utilizes solvents to swell the polyurethane on the film surface. During the swelling process, the solvent gradually penetrates into the polyurethane molecular chains. The penetration depth depends on solvent type and immersion time. Meanwhile, chemical reactions occur as the solvent penetrates the polyurethane molecular chains. Surface crosslinking densifies the polyurethane film structure, making it increasingly difficult for isocyanate to penetrate deeper into the polyurethane film. Therefore, the reaction rate (related to the reaction temperature) will also affect the penetration depth. By optimizing conditions such as solvent type, isocyanate solution concentration, reaction temperature, and reaction time, the penetration depth can be controlled.

Polyurethane with a crosslinked surface structure is difficult to dissolve in solvents. By first forming a film and then modifying its surface, rather than employing a multi-layer coating and curing process, this issue of insolubility associated with crosslinked polyurethane can be avoided.

In Step 2, the isocyanate in the isocyanate solution is one of HMDI, MDI, and HDI, and the solvent is at least one of the following: cyclohexane, toluene, ethyl acetate, and cyclohexanone.

In Step 2, the concentration of the isocyanate solution is 5 wt. %-20 wt. %.

After Step 2 is complete, the prosthetic valve leaflet material undergoes post-treatment, which includes: under an inert gas atmosphere, sequentially rinsing with the solvent of the isocyanate solution, followed by rinsing with diethyl ether, and finally drying.

In Step 2, rinsing with the solvent of the isocyanate solution and with diethyl ether primarily serves to remove residual isocyanate from the polyurethane surface.

A method for preparing a prosthetic valve leaflet material, comprising:
Step 1: preparing a film with a thickness of 80 to 210 μm from polyurethane having an elastic modulus of 5-30 MPa; and
Step 2: applying an isocyanate solution containing a catalyst onto the surface of the film, and reacting at 50-90° C. for 1-3 h; and
Step 3: placing the product of Step 2 in a modifier solution to react for 12-24 h at 30-50° C. to obtain the prosthetic valve leaflet material.

The molecular chain structure of polyurethane includes hard and soft segments, wherein the hard segments include first chain units formed from isocyanate and second chain units formed from a chain extender.

The isocyanate is at least one of the following: toluene diisocyanate (TDI), hexamethylene diisocyanate (HDI), diphenylmethane diisocyanate (MDI), naphthalene diisocyanate (NDI), p-phenylene diisocyanate (PPDI), isophorone diisocyanate (IPDI), xylylene diisocyanate (XDI), and triphenylmethane triisocyanate.

The chain extender is at least one of the following: ethylene glycol, propylene glycol, butanediol, pentanediol, hexanediol, octanediol, ethylenediamine, propylenediamine, butanediamine, pentanediamine, hexanediamine, octanediamine, and 1,3-bis(4-hydroxybutyl)-1,1,3,3-tetramethyldisiloxane (BHTD).

The soft segment raw material is at least one of the following: Dutral T5651, Dutral T5652, polyhexamethylene oxide (PHMO), polytetramethylene glycol (PTMO), adipic acid polyester diol, succinic acid polyester diol and polydimethylsiloxane diol.

In the molecular chain structure of the polyurethane, the isocyanate is one of diphenylmethane diisocyanate, toluene diisocyanate and hexamethylene diisocyanate.

In the molecular chain structure of the polyurethane, the isocyanate is diphenylmethane diisocyanate.

In the molecular chain structure of the polyurethane, the chain extender is at least one of ethylenediamine and 1,3-bis(4-hydroxybutyl)-1,1,3,3-tetramethyldisiloxane.

In the molecular chain structure of the polyurethane, the chain extender includes ethylenediamine and 1,3-bis(4-hydroxybutyl)-1,1,3,3-tetramethyldisiloxane.

In the molecular chain structure of the polyurethane, the chain extender includes ethylenediamine and 1,3-bis(4-hydroxybutyl)-1,1,3,3-tetramethyldisiloxane, and the molar ratio of ethylenediamine to 1,3-bis(4-hydroxybutyl)-1,1,3,3-tetramethyldisiloxane is 1:0.9 to 1:1.1.

In the molecular chain structure of the polyurethane, the soft segment raw material is at least one of polydimethylsiloxane and polyhexamethylene oxide.

In the molecular chain structure of polyurethane, the soft segment raw material includes polydimethylsiloxane and polyhexamethylene oxide.

In the molecular chain structure of the polyurethane, the soft segment raw material includes polydimethylsiloxane and polyhexamethylene oxide, and the mass ratio of polydimethylsiloxane to polyhexamethylene oxide is 3-6:1.

The hard segment content of polyurethane is 30%-60%. The hard segment content of polyurethane is 35% to 60%.

The isocyanate index of polyurethane is 1.0-1.1. The isocyanate index of polyurethane is 1.0-1.05. The isocyanate index of polyurethane is 1.0-1.02.

In Step 2, the isocyanate solution containing a catalyst is applied to the surface of the film, where the isocyanate in the isocyanate solution reacts with the molecular chains of the polyurethane to form a crosslinked structure. Specifically, a single isocyanate molecule chemically bonds with the reactive sites of two polyurethane molecular chains, forming an interlinked structure between the polyurethane molecular chains. The reaction temperature and time for isocyanate side chain crosslinking are relatively high.

The reaction temperature in Step 2 is relatively high. To avoid the same type of chemical reaction from continuing in Step 3, the reaction temperature is lowered. When the reaction temperature is lowered, the reaction between —OH and —NCO will also slow down. To ensure the reaction proceeds sufficiently, the reaction time is extended.

The film in Step 1 can be prepared by using existing techniques, such as dip coating, hot pressing, or other forming methods.

In Step 2, the isocyanate solution containing a catalyst is applied to the surface of the film, allowing the isocyanate to penetrate 5-30 μm from the film surface, and the side chain reaction is initiated by heating to introduce-NCO into the side chain of the polyurethane.

In Step 3, the modification of the polyurethane is achieved by grafting the modifier onto the —NCO group. The introduction of the modifier into the polyurethane, on the one hand, restricts soft segment mobility due to steric hindrance from the side chains, thereby improving the strength and hardness of the polyurethane. Simultaneously, the side chains tend to be distributed on the surface under the guidance of the lowest energy arrangement. The excellent biostability and biocompatibility of the side chains confer biostability and biocompatibility to the polyurethane.

In Step 2, the isocyanate in the isocyanate solution is one of HMDI, MDI, and HDI, and the solvent is one of the following: cyclohexane, toluene, ethyl acetate, and cyclohexanone.

In Step 2, the concentration of the isocyanate solution is 5 wt. % to 20 wt. %.

After Step 2 is complete, the prosthetic valve leaflet material undergoes post-treatment, which includes: under an inert gas atmosphere, sequentially rinsing with the solvent of the isocyanate solution, followed by rinsing with diethyl ether.

In Step 2, rinsing with the solvent of the isocyanate solution and with diethyl ether primarily serves to remove residual isocyanate from the polyurethane surface.

In Step 3, the modifier is one of the following: 3-aminopropyltriethoxysilane, fluorine-containing alcohol (e.g., 4-fluorobenzyl alcohol CAS No.: 459-56-3, 1H, 1H-perfluorododecan-1-ol, CAS No.: 423-65-4), sphingosine phosphorylcholine (CAS No.: 1670-26-4), and 3-((tert-butyldimethylsilyl)oxy)-propanol).

The solvent of the modifier in Step 3 is at least one of the following: cyclohexane, toluene, DMF, DMAc, and DMSO.

A method for preparing a valve leaflet, including:

Step 1: immersing a mold in a first surface-layer polyurethane solution, maintaining it for a predetermined time before removal, and drying it at 30-80° C. under an inert gas atmosphere to form a film.

The above Step 1 is repeated 1 to 9 times to form a first surface layer of the prosthetic valve leaflet material.

Step 2: immersing the mold in an inner-layer polyurethane solution, maintaining it for a predetermined time before removal, and drying it at 30-80° C. under an inert gas atmosphere to form a film.

The above Step 2 is repeated 1 to 9 times to form an inner layer of the prosthetic valve leaflet material.

Step 3: cutting into a leaflet shape and fitting a stent onto the mold.

Step 4, immersing the assembly in a second surface-layer polyurethane solution, taking it out after staying for a predetermined time, and drying it at 30-80° C. under an inert gas atmosphere to form a film.

The above Step 4 is repeated 1 to 9 times to form a second surface layer of the prosthetic valve leaflet material.

Step 5: performing demolding by immersing the mold in water to obtain the valve leaflet.

A method for preparing a valve, including:

Step 1: immersing a mold in a first surface-layer polyurethane solution, maintaining it for a predetermined time before removal, and drying it at 30-80° C. under an inert gas atmosphere to form a film.

The above Step 1 is repeated 1 to 9 times to form a first surface layer of the prosthetic valve leaflet material.

Step 2: immersing the mold in an inner-layer polyurethane solution, maintaining it for a predetermined time before removal, and drying it at 30-80° C. under an inert gas atmosphere to form a film.

The above Step 2 is repeated 1 to 9 times to form an inner layer of the prosthetic valve leaflet material.

Step 3: cutting into a leaflet shape and fitting the stent onto the mold.

Step 4, immersing the assembly in a second surface-layer polyurethane solution, taking it out after staying for a predetermined time, and drying it at 30-80° C. under an inert gas atmosphere to form a film.

The above Step 4 is repeated 1 to 9 times to form a second surface layer of the prosthetic valve leaflet material.

Step 5: performing demolding by immersing the mold in water to obtain the valve.

### Example 1: Introducing Isocyanate Groups into Side Chains and Capping with a Modifier

A method for preparing a prosthetic valve leaflet includes the following Steps:

(1) Preparing a biocompatible polyurethane, with a composition of 44 wt. % PDMS, 11 wt. % PHMO, 31.6 wt. % MDI, 3.2 wt. % BDO, and 10.2 wt. % BHTD.

(2) Preparing 20 wt. % and 10 wt. % polyurethane solutions respectively, using dimethylacetamide (i.e., DMAc) as the solvent.

(3) Slowly immersing a mold in the 20 wt. % polyurethane solution for 5 s.

(4) Drying the mold at 60° C. in a nitrogen atmosphere for 3 h.

(5) Repeating Steps (3) and (4) six times.

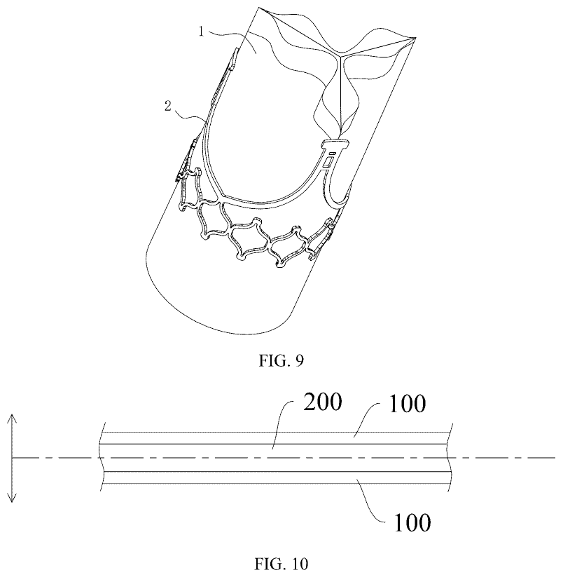

(6) Fitting the stent onto the mold (see FIG. 9, the stent 2 is fitted onto the mold 1), and immersing the mold with the stent in a 10 wt. % polyurethane solution for 5 s.

(7) Drying the assembly at 60° C. under a nitrogen atmosphere for 6 h.

(8) Trimming the valve leaflets, immersing the mold in water for 4 h for demolding (after immersion, the polyurethane separates from the mold, making demolding easy).

(9) Preparing a cyclohexane solution of diphenylmethane-4,4′-diisocyanate (MDI) to form a 10% (w/v) cyclohexane solution.

(10) Under a nitrogen atmosphere, immersing the valve leaflet in a cyclohexane solution containing MDI to react at 70° C. for 2 h (the reaction mechanism is shown below), rinsing the surface of the valve leaflets with cyclohexane solvent and diethyl ether in sequence to remove residual isocyanates, and storing the valve leaflet in a dry nitrogen atmosphere for later use.

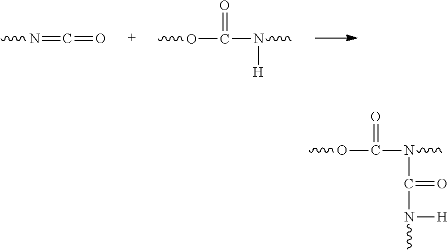

(11) Dissolving 3-Aminopropyltriethoxysilane in dimethylformamide (DMF) to prepare a 5% (w/v) 3-aminopropyltriethoxysilane solution.

(12) Immersing the valve leaflets in 3-aminopropyltriethoxysilane solution to react at 40° C. for 12 h, rinsing the surface several times with the solvent, and then rinsing the surface several times with deionized water.

(13) The valve leaflet has a thickness of 0.19 mm.

### Example 2: Different Chain Extenders

A method for preparing a prosthetic valve leaflet includes the following Steps:

(1) Preparing a surface-layer polyurethane with a composition of 44 wt. % PDMS, 11 wt. % PHMO, 39.2 wt. % MDI, and 5.8 wt. % EDA.

(2) Preparing an inner-layer polyurethane with a composition of 44 wt. % PDMS, 11 wt. % PHMO, 37 wt. % MDI, and 8.0 wt. % BDO.

(3) Preparing a 20 wt. % inner-layer polyurethane solution (using DMAc as solvent), a 20 wt. % surface-layer polyurethane solution (using DMAc as solvent), and a 10 wt. % surface-layer polyurethane solution (using DMAc as solvent).

(4) Slowly immersing a mold in the 20 wt. % surface-layer polyurethane solution for 5 s.

(5) Drying the mold at 60° C. in a nitrogen atmosphere for 3 h.

(6) Slowly immersing the mold in the 20 wt. % inner-layer polyurethane solution for 5 s.

(7) Drying the mold at 60° C. under a nitrogen atmosphere for 3 h.

(8) Repeating Steps (6) and (7) four times.

(9) Fitting a stent onto the mold and immersing the mold with the stent in the 10 wt. % surface-layer polyurethane solution for 5 s.

(10) Drying the assembly at 60° C. under a nitrogen atmosphere for 6 h.

(11) Trimming the valve leaflets, immersing the mold in water for 4 h to demold.

This embodiment features a simple process. Based on the original material system, the chain extender used in the surface-layer polyurethane is adjusted to increase the urea content in the surface-layer polyurethane material, thereby significantly improving the biostability, biocompatibility, creep resistance, and durability of the leaflet.

(12) The valve leaflet has a thickness of 0.18 mm.

### Example 3: Different Contents of Soft and Hard Segments

A method for preparing a prosthetic valve leaflet comprises the following Steps:

(1) Preparing a surface-layer polyurethane with a composition of 44 wt. % PDMS, 11 wt. % PHMO, 32.3 wt. % MDI, 2.3 wt. % EDA, and 10.40 wt. % BHTD.

(2) Preparing an inner-layer polyurethane with a composition of 46.4 wt. % PDMS, 11.6 wt. % PHMO, 30.7 wt. % MDI, 2.0 wt. % EDA, and 9.3 wt. % BHTD.

(3) Preparing a 20 wt. % inner-layer polyurethane solution (using DMAc as solvent), a 20 wt. % surface-layer polyurethane solution (using DMAc as solvent), and a 10 wt. % surface-layer polyurethane solution (using DMAc as solvent).

(4) Slowly immersing a mold in a 20 wt. % surface-layer polyurethane solution for 5 s.

(5) Drying the mold at 60° C. in a nitrogen atmosphere for 3 h.

(6) Slowly immersing the mold in the 20 wt. % inner-layer polyurethane solution for 5 s.

(7) Drying the mold at 60° C. under a nitrogen atmosphere for 3 h.

(8) Repeating Steps (6) and (7) four times.

(9) Fitting the stent onto the mold and immersing the mold with the stent in a 10 wt. % surface-layer polyurethane solution for 5 s.

(10) Drying the assembly at 60° C. under a nitrogen atmosphere for 6 h.

(11) Trimming the valve leaflets, immersing the mold in water for 4 h to demold.

(12) The valve leaflet has a thickness of 0.18 mm.

### Example 4: Surface Crosslinking

A method for preparing a prosthetic valve leaflet includes the following Steps:

(1) Preparing a biocompatible polyurethane with a composition of 44 wt. % PDMS, 11 wt. % PHMO, 31.6 wt. % MDI, 3.2 wt. % BDO, and 10.2 wt. % BHTD.

(2) Preparing a 20 wt. % polyurethane solution (using DMAc as solvent) and 10 wt. % polyurethane solution (using DMAc as solvent).

(3) Slowly immersing a mold in the 20 wt. % polyurethane solution for 5 s.

(4) Drying the mold at 60° C. in a nitrogen atmosphere for 3 h.

(5) Repeating Steps (3) and (4) six times.

(6) Fitting the stent onto the mold and immersing the mold with the stent in a 10 wt. % polyurethane solution for 5 s.

(7) Drying at 60° C. under a nitrogen atmosphere for 6 h.

(8) Trimming the valve leaflets, immersing the mold in water for 4 h to demold.

(9) Preparing a cyclohexane solution of diphenylmethane-4,4′-diisocyanate (MDI) to form a 10% (w/v) cyclohexane solution.

(10) under a nitrogen atmosphere, immersing the valve leaflets in a cyclohexane solution containing MDI at 70° C. for 4 h, rinsing the surface several times with the solvent, and rinsing the surface several times with deionized water.

(11) The valve leaflet surface has a thickness of 0.17 mm.

### Example 5: Different Chain Extenders

A method for preparing a prosthetic valve leaflet includes the following Steps:

(1) Preparing a surface-layer polyurethane with a composition of 44 wt. % PDMS, 11 wt. % PHMO, 39.2 wt. % HDI, and 5.8 wt. % EDA.

(2) Preparing an inner-layer polyurethane with a composition of 44 wt. % PDMS, 11 wt. % PHMO, 37.0 wt. % HDI, and 8.0 wt. % BDO.

(3) Preparing a 20 wt. % inner-layer polyurethane solution (using DMAc as solvent), a 20 wt. % surface-layer polyurethane solution (using DMAc as solvent), and a 10 wt. % surface-layer polyurethane solution (using DMAc as solvent).

(4) Slowly immersing a mold in the 20 wt. % surface-layer polyurethane solution for 5 s.

(5) Drying the mold at 60° C. in a nitrogen atmosphere for 3 h.

(6) Slowly immersing the mold in the 20 wt. % inner-layer polyurethane solution for 5 s.

(7) Drying the mold at 60° C. in a nitrogen atmosphere for 3 h.

(8) Repeating Steps (6) and (7) four times.

(9) Fitting the stent onto the mold and immersing the mold with the stent in the 10 wt. % surface-layer polyurethane solution for 5 s.

(10) Drying at 60° C. under a nitrogen atmosphere for 6 h.

(11) Trimming the valve leaflets, immersing the mold in water for 4 h to demold.

(12) The valve leaflet has a thickness of 0.18 mm.

This example features a simple process. Based on the original material system, the chain extender used in the surface-layer polyurethane is adjusted to increase the urea content in the surface-layer polyurethane material, thereby significantly improving the biostability, biocompatibility, creep resistance, and durability of the leaflet.

### Example 6: Different Chain Extenders

A method for preparing a prosthetic valve leaflet includes the following Steps:

(1) Preparing a surface-layer polyurethane with a composition of 5 wt. % PDMS, 50 wt. % Dutral T5651, 39.2 wt. % MDI, and 5.8 wt. % EDA.

(2) Preparing an inner-layer polyurethane with a composition of 5 wt. % PDMS, 50 wt. % Dutral T5651, 37.0 wt. % MDI, and 8.0 wt. % BDO.

(3) Preparing a 20 wt. % inner-layer polyurethane solution (using DMAc as solvent), a 20 wt. % surface-layer polyurethane solution (using DMAc as solvent), and a 10 wt. % surface-layer polyurethane solution (using DMAc as solvent).

(4) Slowly immersing a mold in the 20 wt. % surface-layer polyurethane solution for 5 s.

(5) Drying the mold at 60° C. in a nitrogen atmosphere for 3 h.

(6) Slowly immersing the mold in the 20 wt. % inner-layer polyurethane solution for 5 s.

(7) Drying the mold at 60° C. under a nitrogen atmosphere for 3 h.

(8) Repeating Steps (6) and (7) four times.

(9) Fitting the stent onto the mold and immersing the mold with the stent in the 10 wt. % surface-layer polyurethane solution for 5 s.

(10) Drying at 60° C. under a nitrogen atmosphere for 6 h.

(11) Trimming the valve leaflets, immersing the mold in water for 4 h to demold.

(12) The valve leaflet has a thickness of 0.18 mm.

This example features a simple process. Based on the original material system, the chain extender used in the surface-layer polyurethane is adjusted to increase the urea content in the surface-layer polyurethane material, thereby significantly improving the biostability, biocompatibility, creep resistance, and durability of the leaflet.

### Example 7: Surface Layer with Two Sub-Layers

A method for preparing a prosthetic valve leaflet includes the following Steps:

(1) Preparing a first sub-layer of the surface-layer polyurethane, wherein the surface-layer polyurethane has a composition of 5 wt. % PDMS, 50 wt. % Dutral T5651, 39.2 wt. % MDI, and 5.8 wt. % EDA.

(2) Preparing a second sub-layer of the surface-layer polyurethane, wherein the composition of the surface-layer polyurethane is 4.4 wt. % PDMS, 44 wt. % Dutral T5651, 44.8 wt. % MDI, and 6.8 wt. % EDA.

(3) Preparing an inner-layer polyurethane with a composition of 5 wt. % PDMS, 50 wt. % Dutral T5651, 37.0 wt. % MDI, and 8.0 wt. % BDO.

(4) Preparing a 20 wt. % inner-layer polyurethane solution (using DMAc as the solvent), an 8 wt. % surface-layer polyurethane solution for the first sub-layer (using DMAc as the solvent), an 8 wt. % surface-layer polyurethane solution for the second sub-layer (using DMAc as the solvent), a 15 wt. % surface-layer polyurethane solution for the first sub-layer (using DMAc as the solvent), and a 15 wt. % surface-layer polyurethane solution for the second sub-layer (using DMAc as the solvent).

(5) Slowly immersing a mold in the 15 wt. % surface-layer polyurethane solution for the second sub-layer for 5 s.

(6) Drying the mold at 60° C. in a nitrogen atmosphere for 3 h.

(7) Slowly immersing the mold in the 15 wt. % surface first sub-layer polyurethane solution for 5 s.

(8) Drying the mold at 60° C. in a nitrogen atmosphere for 3 h.

(9) Slowly immersing the mold in the 20 wt. % inner-layer polyurethane solution for 5 s.

(10) Drying the mold at 60° C. under a nitrogen atmosphere for 3 h.

(11) Repeating Steps (9) and (10) four times.

(12) Fitting the stent onto the mold, and immersing the mold with the stent in the 8 wt. % surface-layer polyurethane solution for the first sub-layer for 5 s.

(13) Drying the mold at 60° C. under a nitrogen atmosphere for 3 h.

(14) Immersing the mold with the stent in the 8 wt. % surface-layer polyurethane solution for the second sub-layer for 5 s.

(15) Drying the mold at 60° C. under a nitrogen atmosphere for 6 h.

(16) Trimming the valve leaflets, immersing the mold in water for 4 h to demold.

(17) The valve leaflet has a thickness of 0.18 mm.

### Comparative Example 1

A method for preparing a prosthetic valve leaflet includes the following Steps:

(1) Preparing a polyurethane with a composition of 44 wt. % PDMS, 11 wt. % PHMO, 32.3 wt. % MDI, 2.3 wt. % EDA, and 10.40 wt. % BHTD.

(2) Preparing a 20 wt. % polyurethane solution (using DMAc as solvent) and 10 wt. % polyurethane solution (using DMAc as solvent).

(3) Slowly immersing the mold in the 20 wt. % polyurethane solution for 5 s.

(4) Drying a mold at 60° C. in a nitrogen atmosphere for 3 h.

(5) Repeating Steps (3) and (4) five times.

(6) Fitting the stent onto the mold and immersing the mold with the stent in the 10 wt. % polyurethane solution for 5 s.

(7) Drying the assembly at 60° C. under a nitrogen atmosphere for 6 h.

(8) Trimming the valve leaflets, immersing the mold in water for 4 h to demold.

(9) The valve leaflet has a thickness of 0.18 mm.

### Comparative Example 2

A method for preparing a prosthetic valve leaflet includes the following Steps:

(1) Preparing a polyurethane with a composition of 44 wt. % PDMS, 11 wt. % PHMO, 31.6 wt. % MDI, 3.2 wt. % BDO, and 10.2 wt. % BHTD.

(2) Preparing a 20 wt. % polyurethane solution (using DMAc as solvent) and 10 wt. % polyurethane solution (using DMAc as solvent).

(3) Slowly immersing a mold in 20 wt. % polyurethane solution for 5 s.

(4) Drying the mold at 60° C. in a nitrogen atmosphere for 3 h.

(5) Repeating Steps (3) and (4) five times.

(6) Fitting the stent onto the mold and immersing the mold with the stent in a 10 wt. % polyurethane solution for 5 s.

(7) Drying at 60° C. under a nitrogen atmosphere for 6 h.

(8) Trimming the valve leaflets, immersing the mold in water for 4 h to demold.

(9) The valve leaflet has a thickness of 0.18 mm.

### Performance Characterization

The performance characterization results of Examples 1-7 and Comparative Examples 1-2 are shown in Table 1.

TABLE 1

| | | | | Trans- | |
|---|---|---|---|---|---|
| | | | | valvular | |
| | Hydrolysis | Oxidation | Fatigue | Gradient | EOA |
| | resistance | resistance | resistance | (MPa) | (mm²) |
| Example 1 | excellent | excellent | No damage | 10.89 | 1.80 |
| | | | after 600 | | |
| | | | million | | |
| | | | cycles | | |
| Example 2 | excellent | excellent | No damage | 10.54 | 1.79 |
| | | | after 600 | | |
| | | | million | | |
| | | | cycles | | |
| Example 3 | excellent | excellent | No damage | 10.67 | 1.84 |
| | | | after 600 | | |
| | | | million | | |
| | | | cycles | | |
| Example 4 | excellent | excellent | No damage | 12.02 | 1.76 |
| | | | after 600 | | |
| | | | million | | |
| | | | cycles | | |
| Example 5 | excellent | excellent | No damage | 13.01 | 1.74 |
| | | | after 600 | | |
| | | | million | | |
| | | | cycles | | |
| Example 6 | excellent | excellent | No damage | 13.12 | 1.72 |
| | | | after 600 | | |
| | | | million | | |
| | | | cycles | | |
| Example 7 | excellent | excellent | No damage | 12.07 | 1.87 |
| | | | after 600 | | |
| | | | million | | |
| | | | cycles | | |
| Comparative | excellent | excellent | No damage | 17.89 | 1.50 |
| Example 1 | | | after 600 | | |
| | | | million | | |
| | | | cycles | | |
| Comparative | poor | poor | Tearing | 10.83 | 1.83 |
| Example 2 | | | occurred at | | |
| | | | the junction | | |
| | | | of stent and | | |
| | | | valve leaflet | | |
| | | | after 51.8 | | |
| | | | million | | |
| | | | cycles | | |

The test methods for hydrolysis resistance and oxidation resistance are as follows:

The valve leaflet material is maintained at 150% strain in a solution of 20 wt. % H₂O₂ and 0.1 mol/L CoCl₂ (pH=5.0±0.2) at 37° C. for 90 days. If no significant changes in surface morphology occur, it is considered to have excellent oxidation resistance. Superior oxidation resistance also reflects good biostability to a certain extent. Good oxidation resistance means that defects are less likely to form on the material surface, which can, to some extent, ensure fatigue resistance.

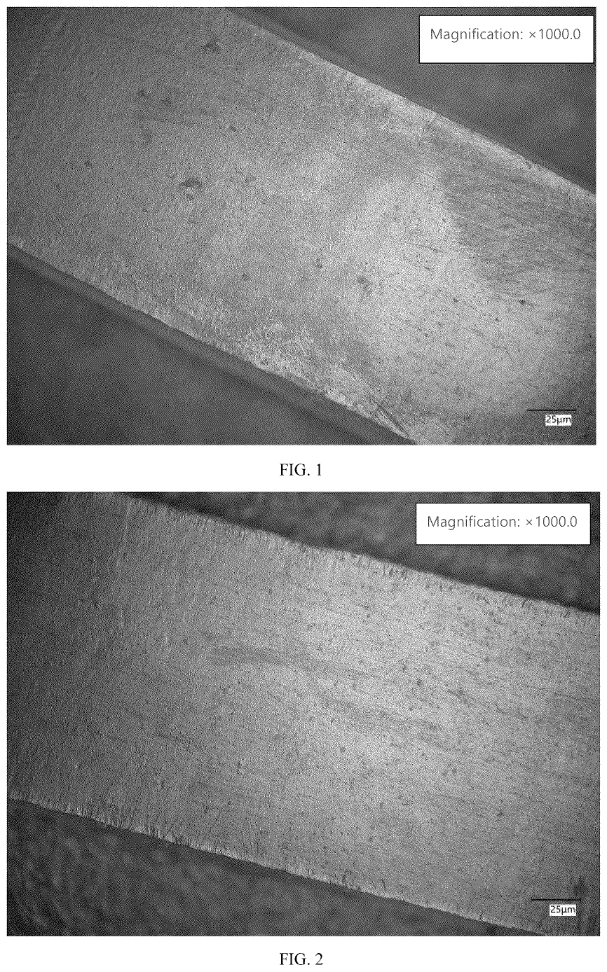

In Example 1, isocyanate was introduced into the polyurethane side chains on the valve leaflet surface and capped with a modifier, achieving leaflet surface modification. According to the oxidation-resistance test results, Example 1 (see FIG. 1) exhibited a smooth surface without oxidation traces, whereas Comparative Example 2 (see FIG. 8), which did not undergo surface modification, exhibited a relatively large number of cracks. This demonstrates that the introduction of isocyanate into the polyurethane side chains on the valve leaflet surface and capping with a modifier can significantly improve the valve leaflet's biostability and fatigue performance, which is related to the reduced likelihood of surface defects after modification. In contrast, the transvalvular pressure gradient and EOA remained unchanged, demonstrating that surface modification had little effect on the valve leaflet's hydrodynamic performance.

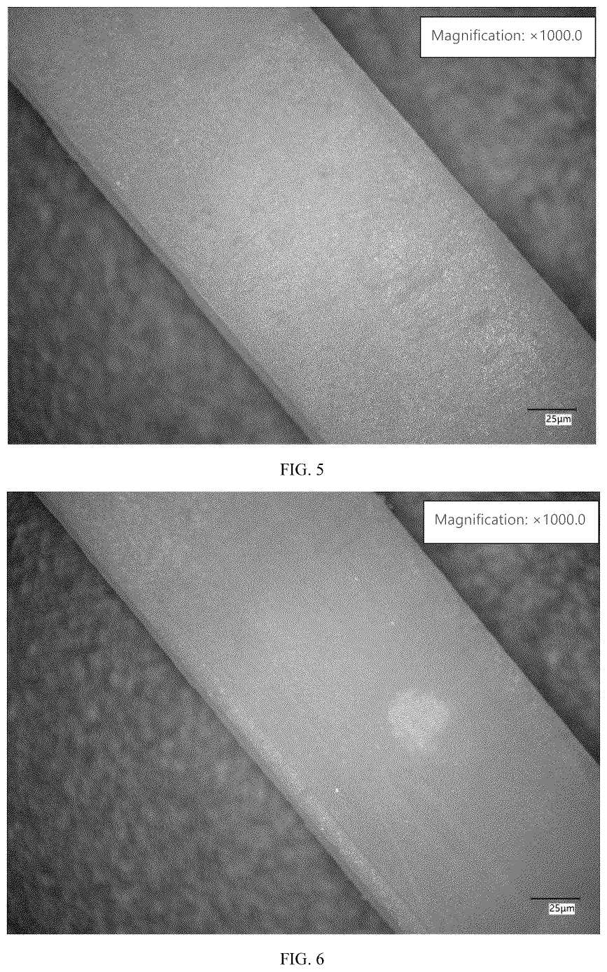

The surface layer and inner layer in Examples 2, 5, and 6 utilized polyurethane materials with different chain extenders. The surface-layer polyurethane incorporated amines as chain extenders, enhancing the interaction forces between hard and soft segments. This results in a denser overall structure, improving its inherent biostability while providing protective effects for the inner layer. Additionally, the strengthened interaction forces between hard and soft segments reduces the mobility of soft segments, thereby diminishing the transport of water vapor and oxygen into the interior, further protecting the inner-layer polyurethane. The results of the oxidation-resistance test show that the surfaces of Example 2 (see FIG. 2), Example 5 (see FIG. 5), and Example 6 (see FIG. 6) were smooth without obvious cracks, indicating a significant improvement in their surface oxidation resistance, and fewer surface defects further led to a significant enhancement in fatigue performance. In contrast, the transvalvular pressure gradient and effective orifice area (EOA) show no significant changes, indicating that the surface-distributed polyurethane coating had minimal impact on the hydrodynamic performance of the valve leaflet.

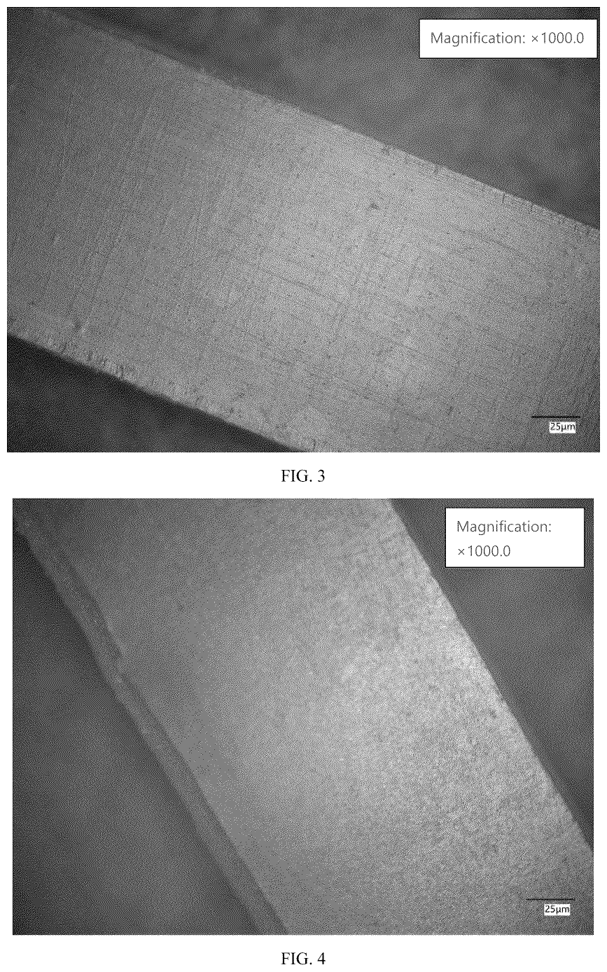

In Example 3, a polyurethane with a higher hard segment content is used in the surface layer. A higher hard segment content means each soft segment experiences greater force from hard segments, resulting in a denser overall structure. This enhances the material's inherent biostability, reduces the mobility of soft segments, and diminishes the ability of water and oxygen to penetrate inward, thereby protecting the inner-layer polyurethane. At the same time, since the soft segments are the main target of oxidative attack on polyurethane, a higher proportion of hard segments corresponds to better biostability, which further improves the biostability of the surface layer. Example 3 (see FIG. 3) shows minor surface cracks, but the cracks are much shallower than those in Comparative Example 2, demonstrating a significant improvement in the biostability of the leaflets. The reduced tendency of surface defect formation also leads to a great enhancement in fatigue performance. In contrast, the transvalvular pressure gradient and EOA show no significant changes, demonstrating that the distributed surface-layer polyurethane has almost no effect on the leaflet's hydrodynamic performance.

In Example 4, the polyurethane surface was modified by crosslinking. After crosslinking, chemical bonds form between the molecular chains of polyurethane, significantly restricting relative movement between soft segment molecules. This enhances the overall density of the soft segments, improving their inherent biostability. As the mobility of soft segments decreases, the ability of water and oxygen to penetrate inward is reduced, thereby protecting the inner-layer polyurethane. Compared with Comparative Example 2 (see FIG. 8), Example 4 (see FIG. 4) exhibits a smooth, defect-free surface, which demonstrates a significant improvement in the valve leaflet's biostability, as its surface is less prone to defects, thereby substantially enhancing fatigue performance. In contrast, the transvalvular pressure gradient and effective orifice area (EOA) show no significant changes, indicating that the distributed surface-layer polyurethane has almost no effect on the leaflet's hydrodynamic performance.

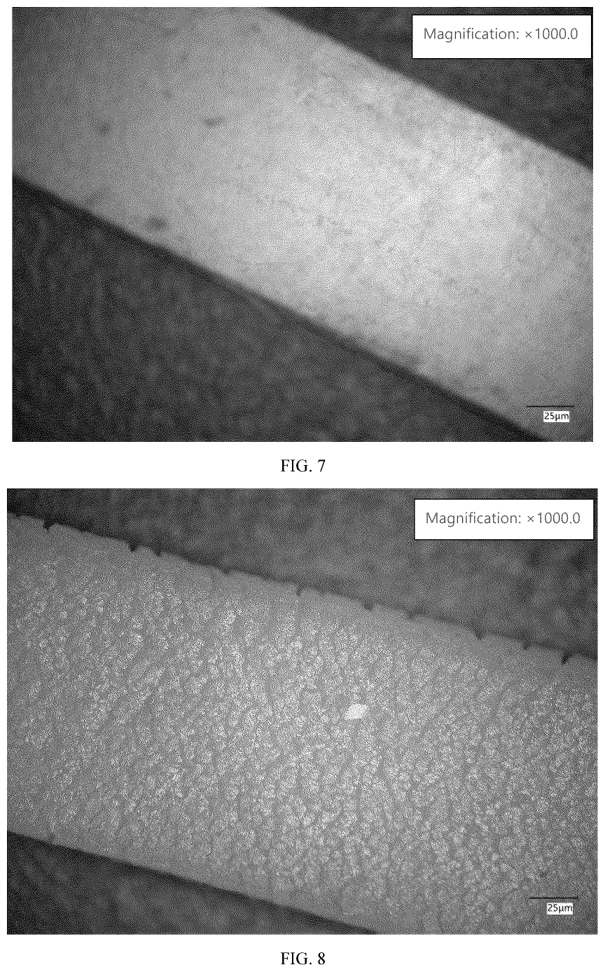

Comparative Example 1 (see FIG. 7) uses materials with an overall higher elastic modulus. Based on the oxidation-resistance test results, its surface is very smooth, leading to significantly enhanced overall biostability and creep resistance. However, due to its increased overall hardness, the hydrodynamic performance is significantly reduced.

The technical features of the above embodiments can be arbitrarily combined, and not all possible combinations of the technical features of the above embodiments have been described for the sake of brevity of description. However, as long as there is no contradiction in the combination of these technical characteristics, such combination should be regarded as falling into the scope of this specification. When technical features of different embodiments are embodied in the same drawing, it can be deemed that the drawing also discloses examples of combinations of the various embodiments involved.

The above-described embodiments only illustrate several embodiments of the present disclosure, and the descriptions thereof are specific and detail, but should not be construed as limiting the scope of the patent disclosure. It should be noted that, for those of ordinary skill in the art, several modifications and improvements can be made without departing from the concept of the present disclosure, all of which fall into the protection scope of the present disclosure.

What is claimed is:

1. A prosthetic valve leaflet material made of polyurethane, comprising, in a thickness direction:
an inner layer; and
surface layers disposed on two opposite sides of the inner layer,
wherein elastic modulus of surface-layer polyurethane is greater than elastic modulus of inner-layer polyurethane.

2. The prosthetic valve leaflet material according to claim 1, wherein one or more of the following conditions is satisfied:
a. the elastic modulus of the surface-layer polyurethane is 10 MPa to 60 MPa greater than the elastic modulus of the inner-layer polyurethane;
b. oxygen permeability of the surface-layer polyurethane is not more than 9*10⁹ (cm²/(s*cm*Hg)).

3. The prosthetic valve leaflet material according to claim 1, wherein the elastic modulus of the surface-layer polyurethane ranges from 30 MPa to 100 MPa, and the elastic modulus of the inner-layer polyurethane ranges from 5 MPa to 30 MPa.

4. The prosthetic valve leaflet material according to claim 1, wherein molecular chain structures of the surface-layer polyurethane and the inner-layer polyurethane satisfy one or more of the following conditions:
a. hard segment content of the inner-layer polyurethane is lower than that of the surface-layer polyurethane;
b. the inner-layer polyurethane is prepared using an alcohol-based chain extender, while the surface-layer polyurethane is prepared using an amine-containing chain extender;
c. the surface-layer polyurethane has a higher crosslink density than the inner-layer polyurethane.

5. The prosthetic valve leaflet material according to claim 1, wherein molecular chain structures of the surface-layer polyurethane and the inner-layer polyurethane each comprise hard and soft segments, and the hard segments comprise first chain units formed from isocyanate and second chain units formed from a chain extender;
wherein one or more of the following conditions is satisfied:
a. the isocyanate is at least one selected from a group consisting of toluene diisocyanate (TDI), hexamethylene diisocyanate (HDI), diphenylmethane diisocyanate (MDI), naphthalene diisocyanate (NDI), p-phenylene diisocyanate (PPDI), isophorone diisocyanate (IPDI), xylylene diisocyanate (XDI), and triphenylmethane triisocyanate;
b. the chain extender is at least one selected from a group consisting of ethylene glycol, propylene glycol, butanediol, pentanediol, hexanediol, octanediol, ethylenediamine, propylenediamine, butanediamine, pentanediamine, hexanediamine, octanediamine, and 1,3-bis(4-hydroxybutyl)-1,1,3,3-tetramethyldisiloxane (BHTD);
c. a soft segment raw material is at least one selected from a group consisting of Dutral T5651, Dutral T5652, polyhexamethylene oxide (PHMO), polytetramethylene glycol (PTMO), adipic acid polyester diol, succinic acid polyester diol, and polydimethylsiloxane diol.

6. The prosthetic valve leaflet material according to claim 1, wherein one or more of the following conditions is satisfied:
a. hard segment content of each of the surface-layer polyurethane and the inner-layer polyurethane ranges from 30% to 60%;
b. an isocyanate index of each of the surface-layer polyurethane and the inner-layer polyurethane ranges from 1.0 to 1.02.

7. The prosthetic valve leaflet material according to claim 1, wherein one or more of the following conditions is satisfied:
a. a thickness of the prosthetic valve leaflet material ranges from 75 μm to 210 μm;
b. a thickness of the inner-layer polyurethane ranges from 70 μm to 170 μm;
c. a thickness of the surface-layer polyurethane ranges from 5 μm to 40 μm.

8. The prosthetic valve leaflet material according to claim 1,
wherein the inner layer comprises at least one first sub-layer along the thickness direction, wherein either each first sub-layer is made of an identical type of polyurethane, or, from inside to outside along the thickness direction, the elastic modulus of each first sub-layer gradually increases by a predetermined interval; or
the surface layer comprises at least one second sub-layer along the thickness direction, wherein either each second sub-layer is made of an identical type of polyurethane, or, from the inside to the outside along the thickness direction, the elastic modulus of each second sub-layer gradually increases by a predetermined interval.

9. The prosthetic valve leaflet material according to claim 1, wherein one or more of the following conditions is satisfied:
a. the hard segment content of the surface-layer polyurethane ranges from 45% to 60%;
b. the hard segment content of the inner-layer polyurethane ranges from 30% to 45%.

10. The prosthetic valve leaflet material according to claim 1, wherein hard segments in molecular chain structures of the surface-layer polyurethane and the inner-layer polyurethane are each modified in one of the following ways:
A. introducing fluorine-containing side chains;
B. introducing a chain extender containing POSS groups; and
wherein introduction of the fluorine-containing side chains is achieved by adding a fluorine-containing diisocyanate with a mass fraction of 3% to 10% to synthetic raw materials of the polyurethane; and
wherein introduction of the POSS groups is achieved by adding a POSS-containing diol chain extender with a mass fraction of 1% to 3% to the synthetic raw materials of the polyurethane.

11. The prosthetic valve leaflet material according to claim 1, wherein the surface-layer polyurethane is subjected to crosslinking modification, and the crosslinking modification of the surface-layer polyurethane is achieved via either irradiation crosslinking or chemical crosslinking.

12. A valve leaflet made of a prosthetic valve leaflet material according to claim 1.

13. A valve, comprising:
a stent defining a blood flow channel therein; and
one or more valve leaflets made of the prosthetic valve leaflet material according to claim 1.

14. A method for preparing a prosthetic valve leaflet material according to claim 1, comprising:
Step 1: immersing a mold in a first surface-layer polyurethane solution, maintaining the mold for a predetermined time before removal, and drying the mold at 30° C. to 80° C. under an inert gas atmosphere to form a film;
wherein Step 1 is repeated 1 to 9 times to form a first surface layer of the prosthetic valve leaflet material;
Step 2: immersing the mold in an inner-layer polyurethane solution, maintaining the mold for a predetermined time before removal, and drying the mold at 30° C. to 80° C. under the inert gas atmosphere to form a film;
wherein Step 2 is repeated 1 to 9 times to form an inner layer of the prosthetic valve leaflet material;
Step 3: fitting a stent onto the mold, immersing the mold in a second surface-layer polyurethane solution, maintaining the mold for a predetermined time before removal, and drying the mold at 30° C. to 80° C. under the inert gas atmosphere to form a film;
wherein Step 3 is repeated 1 to 9 times to form a second surface layer of the prosthetic valve leaflet material; and
Step 4: performing demolding by immersing the mold in water to obtain the prosthetic valve leaflet material.

15. The method according to claim 14, wherein a concentration of the first surface-layer polyurethane solution ranges from 10% to 30% (w/v), a concentration of the second surface-layer polyurethane solution ranges from 5% to 15% (w/v), and a concentration of the inner-layer polyurethane solution ranges from 10% to 30% (w/v); and
wherein a solvent of each of the first surface-layer polyurethane solution, the second surface-layer polyurethane solution, and the inner-layer polyurethane solution is one selected from a group consisting of N,N-dimethylacetamide (DMAc), dimethylformamide (DMF), dimethyl sulfoxide (DMSO), toluene, and tetrahydrofuran (THF).

16. The method according to claim 14, wherein in Step 1, a drying time is 1 h to 6 h, and a predetermined maintaining time in the first surface-layer polyurethane solution ranges from 5 s to 10 s;
wherein in Step 2, a predetermined maintaining time in the inner-layer polyurethane solution ranges from 5 s to 10 s; and
wherein in Step 3, a predetermined maintaining time in the second surface-layer polyurethane solution ranges from 5 s to 10 s.

17. A method for preparing a prosthetic valve leaflet material according to claim 1, comprising:
Step 1: preparing a film with a thickness of 80 μm to 210 μm from polyurethane having an elastic modulus of 5 MPa to 30 MPa; and
Step 2: applying an isocyanate solution onto a surface of the film, and allowing the isocyanate solution to react with the film at 50° C. to 90° C. for 3 h to 5 h to obtain the prosthetic valve leaflet material;
wherein in Step 2, an application method comprises at least one of immersion, coating, and spraying;
wherein isocyanate penetrates 5 μm to 30 μm from the surface of the film, and heating is then performed to initiate side-chain crosslinking; and
wherein the isocyanate in the isocyanate solution is one selected from HMDI, MDI, and HDI, a solvent of the isocyanate solution is one selected from a group consisting of cyclohexane, toluene, ethyl acetate, and cyclohexanone, and a concentration of the isocyanate solution ranges from 5 wt. % to 20 wt. %.

18. A method for preparing a prosthetic valve leaflet material according to claim 1, comprising:
Step 1: preparing a film with a thickness of 80 μm to 210 μm from polyurethane having an elastic modulus of 5 MPa to 30 MPa;
Step 2: applying an isocyanate solution onto a surface of the film, and allowing the isocyanate solution to react with the film at 50° C. to 90° C. for 1 h to 3 h; and
Step 3: placing a product of Step 2 into a modifier solution, and allowing the product and the modifier solution to react at 30° C. to 50° C. for 12 h to 24 h to obtain the prosthetic valve leaflet material.

19. The method according to claim 18, wherein in Step 2, isocyanate in the isocyanate solution is one selected from HMDI, MDI, and HDI, a solvent of the isocyanate solution is one selected from a group consisting of cyclohexane, toluene, ethyl acetate, and cyclohexanone, and a concentration of the isocyanate solution ranges from 5 wt. % to 20 wt. %; and
wherein in Step 3, a modifier is one selected from a group consisting of 3-aminopropyltriethoxysilane, fluorine-containing alcohol, sphingosine phosphorylcholine, and 3-((tert-butyldimethylsilyl)oxy)-propanol, and a solvent of the modifier solution is at least one selected from a group consisting of cyclohexane, toluene, DMF, DMAc, and DMSO.

20. A method for preparing a valve leaflet according to claim 12, comprising:
Step 1: immersing a mold in a first surface-layer polyurethane solution, maintaining the mold for a predetermined time before removal, and drying the mold at 30° C. to 80° C. under an inert gas atmosphere to form a film;
wherein Step 1 is repeated 1 to 9 times to form a first surface layer of a prosthetic valve leaflet material;
Step 2: immersing the mold in an inner-layer polyurethane solution, maintaining the mold for a predetermined time before removal, and drying the mold at 30° C. to 80° C. under the inert gas atmosphere to form a film;
wherein Step 2 is repeated 1 to 9 times to form an inner layer of the prosthetic valve leaflet material;
Step 3: cutting the material into a valve leaflet shape and fitting a stent onto the mold;
Step 4: immersing the mold in a second surface-layer polyurethane solution, maintaining the mold for a predetermined time before removal, and drying the mold at 30° C. to 80° C. under the inert gas atmosphere to form a film;
wherein Step 4 is repeated 1 to 9 times to form a second surface layer of the prosthetic valve leaflet material; and
Step 5: performing demolding by immersing the mold in water to obtain the valve leaflet.
